SEQUENCE LISTING (5 sequences)

SEQ ID NO: 1 - protein, 347 aa, Streptococcus pneumoniae
MEKKKLRINMLSSSEKVAGQGVSGAYRELVRLLHRAAKDQLIVTENLPIEADVTHFHTIDFPYYLSTFQKKRSGRKIGYVHFLPATLEGSLKIPFFLKGIVKRYVFSFYNRMEHLVVVNPMFIEDLVAAGIPREKVTYIPNFVNKEKWHPLPQEEVVRLRTDLGLSDNQFIVVGAGQVQKRKGIDDFIRLAEELPQITFIWAGGFSFGGMTDGYEHYKKIMENPPKNLIFPGIVSPERMRELYALADLFLLPSYNELFPMTILEAASCEAPIMLRDLDLYKVILEGNYRATAGREEMKEAILEYQANPAVLKDLKEKAKNISREYSEEHLLQIWLDFYEKQAALGRK

SEQ ID NO: 2 - protein, 347 aa, Streptococcus mitis
MEKKKLRINMLSSSEKVAGQGVSGAYRELVRLLHRDAKDQLIVTENLPIEADVTHFHTIDFPYYLSTFQKKRSGRKIGYVHFLPDTLEGSLKIPFFLKGIVKRYVFSFYNRMEHLVVVNPMFIEDLVAAGIPREKVTYIPNFVNKEKWHPLPQEEVARLRTDLGLSDNQFIVVGAGQVQKRKGIDDFIRLAEELPQITFIWAGGFSFGGMTDGYERYKKIMENPPKNLIFPGIVSPERMRELYALADLFLLPSYNELFPMTILEAASCEAPIMLRDLDLYKVILEGNYRATADREEMKEAILEYQANPAALKDLKEKAKNISREYSEEHLLQIWLDFYEKQAALGRK

SEQ ID NO: 3 - protein, 377 aa, Streptococcus oralis
MEKRGNFPLFSSSLSFLLLILFKENDIIVVMEKKKLRINMLSSSEKVAGQGVSGAYRELVRLLHRDAKDQLIVTENLPIEADVTHFHTIDFPYYLSTFQKKRSGRKIGYVHFLPDTLEGSLKIPFFLKGIVKRYVFSFYNRMEHLVVVNPMFIEDLVAAGIPREKVTYIPNFVNKEKWHPLPQEEVARLRTELGLSDNQFIVVGAGQVQKRKGIDDFIRLAEELPEITFIWAGGFSFGGMTDGYERYKKIMENPPKNLIFPGIVSPEQMRELYALADLFLLPSYNELFPMTILEAASCEAPIMLRDLDLYKVILEGNYRATADREEMKEAILEYQANPAALKDLKEKAKNISREYSEEHLLQIWLDFYEKQDALGRK

SEQ ID NO: 4 - protein, 345 aa, Streptococcus hyointestinalis
MTKEKIKVNMLSSSETVPGQGVSGAYRELMRLLKRDAREAIDLTENKRIKADITHYHTIDIPFYLSTFRKKRVGRRIGYVHFLPDTLQGSLRIPRIVQPIVAKYVTSFYNRMDHLVVVNPSFIEDLVAFGIDRDKITYIPNFVNSEKWFKPSAEDKSAVRKRYGWKDEDFVVLGAGQIQKRKGIDDFVTLAKTNPDIQFVWAGGFSFGGMTDGYDAYKKLMKNPPANLTFTGIVDADEMRQLYGACDLFLLPSFNELFPMTILEAAACGAPIMLRDLELYRIILEGMYLPASDVSKMSEAIRHLRTHPTELNHLQSQSEKISETYSEERLLETWLDFYRQQADLK

SEQ ID NO: 5 - protein, 347 aa, Streptococcus suis
MSTKKLVINMLSSSEKVKGQGVSGAYRELMHLLRERGTTKLDIQEKLFVKADVTHYHTIDPIFFLTTFFKKRTGRRVGYVHFLPDTLEGSLQIPRFLTPLVSWYVITFYNRMDQLVVVNPSFIDDLVEYGISRDKITYLPNFVNRDQWHPLSREEVQDVRKHYAISPNQFVVLGAGQVQRRKGIDDFAQLARECPDVTFVWAGGFSFGNMTDGHAEYKKLMEQPPKNLVFTGIVEAEEMLKLYNMADVFFLPSYSELFPMTILEAASCGTAIMLRDIDLYKVILEGMYLPVKNMDEMKTAIETLQSQPNQLVELTNKAGEISNYYSPEALLEKWLAFYQEQADLAKS

(19) United States
(12) Patent Application Publication
(10) Pub. No.: US 2025/0000962 A1
(43) Pub. Date: Jan. 2, 2025

(54) PNEUMOCOCCAL POLYPEPTIDE ANTIGENS

(71) Applicants: UNIVERSITÉ DE LAUSANNE, Lausanne (CH); INSERM (INSTITUT NATIONAL DE LA SANTE ET DE LA RECHERCHE MEDICALE), Paris (FR); CENTRE NATIONAL DE LA RECHERCHE SCIENTIFIQUE, Paris (FR); INSTITUT PASTEUR DE LILLE, Lille (FR); UNIVERSITE DE LILLE, Lille (FR); CENTRE HOSPITALIE RREGIONAL UNIVERSITAIRE DE LILLE, Lille (FR)

(72) Inventors: Jan-Willem VEENING, Preverenges (CH); Xue LIU, Renens (CH); Jean-Claude SIRARD, Villeneuve d'Ascq (FR); Florian Patrick BOCK, Lausanne (CH); Laurye VAN MAELE, Leers (FR)

(21) Appl. No.: 18/292,376

(22) PCT Filed: Jul. 27, 2022
(86) PCT No.: PCT/EP2022/071098

§ 371 Date: Jan. 26, 2024

(30) Foreign Application Priority Data
Jul. 29, 2021 (EP) 21188570.2

(51) Int. Cl. A61K 39/09; A61K 9/00; A61K 39/00; A61P 31/04

(52) U.S. Cl. CPC A61K 39/092; A61K 9/0019; A61K 9/0043; A61P 31/04; A61K 2039/53; A61K 2039/543; A61K 2039/545; A61K 2039/55505; A61K 2039/55516; A61K 2039/572; A61K 2039/6087

(57) ABSTRACT

The present invention provides novel pneumococcal poly peptide antigens and nucleic acids encoding such antigens and compositions that can be used as vaccines to prevent disease caused by Streptococcus pneumoniae.

### FIELD OF THE INVENTION

The present invention provides novel pneumococcal polypeptide antigens and nucleic acids encoding such antigens and compositions that can be used as vaccines to prevent disease caused by Streptococcus pneumoniae.

### BACKGROUND OF THE INVENTION

Streptococcus pneumoniae is an important human pathogen, which causes severe invasive diseases, like pneumonia, bacteremia, and meningitis. The infectious diseases caused by S. pneumoniae lead to more than one million of death every year (O'Brien et al., 2009). S. pneumoniae resides in the nasopharynx and is carried in over 95% of newborns within the first two months of life and is generally present in about 40% of the human population.

The currently available vaccines for S. pneumoniae disease are all capsule-polysaccharide (CPS) based, for example, Pfizer Prevnar 13R, which is a PCV (pneumococcal conjugate vaccine) containing thirteen different CPSs conjugated to a carrier protein, and Merck Pneumovax, which contains 23 different CPSs. PCVs have a major advantage over solely CPS-based vaccines as they also induce a T-cell dependent immune response (Avci et al., 2011). While CPS and PCVs are extremely successful in reducing the burden of disease caused by serotypes included in these vaccines, theses vaccines provide no prevention of pneumococcal diseases caused by serotypes not included in these vaccines. There are more than 100 known serotypes of S. pneumoniae (Ganaie et al., 2020), however, the CPS-based vaccines only cover a small part. For example, the broadest CPS-based vaccine currently approved only covers 23 serotypes (PneumoVax). In addition, rapid switching between serotypes, serotype displacement and appearance of non-typeable clinical isolates further reduces the protectivity of the CPS-based vaccines and PCVs (Scelfo et al., 2021). Finally, including more polysaccharides in Prevnar 7® to Prevnar 13®, reduced the individual immune response to each individual CPS. This is likely caused by the increased carrier/polysaccharide burden, so-called carrier-induced suppression (Pobre et al., 2014). For instance, Prevnar uses CRM197 as protein carrier/adjuvant, which is a non-toxic diphtheria toxin mutant, and by including more polysaccharides, more of the immune response is targeted to CRM197 instead of the pneumococcal capsules.

One promising approach to reduce carrier-induced suppression is by the incorporation of non-native amino acids (nnAA) as conjugation anchors on the protein carrier avoiding T-cell epitopes thereby using less protein carrier while still retaining efficacy (https://vaxcyte.com/pipeline/). Another recent approach is by directly labeling the CPS with biotin and fusing surface-exposed pneumococcal proteins with a biotin-binding protein, which will then form a tightly bound PCV complex (Zhang et al., 2013). While these approaches might enable higher valent PCVs than currently available, serotype escape is likely to also render these vaccines ineffective on the long term. In addition, S. pneumoniae poses a great threat to global public health in combination with viral infection. One example is the catastrophic influenza A virus (IAV) pandemic of 1918, where severe pneumococcal infections occurred in the aftermath of IAV infection and contributed significantly to excess morbidity and mortality (McCullers, 2014). During the COVID-19 pandemic, there are reports showing the coinfection of S. pneumoniae increased the morbidity and mortality (O'Toole, 2021). Unfortunately, the CPS-based vaccines, including PneumoVax and Prevnar, provide poor protection during the superinfection caused by virus and bacteria coinfection (Jirru et al., 2020; Metzger et al., 2015). In general, PCVs have as goal to reduce pneumococcal colonization and thereby invasive pneumococcal disease (IPD) and the spread and evolution of new strain variants. However, on a longer term, an ideal pneumococcal vaccine would not alter commensal colonization but prevent IPD, as total eradication of the pneumococcal reservoir might enable other opportunistic pathogens to occupy that niche (Gonzalez and Jacobs, 2013).

One promising avenue is in the use of immunogenic pneumococcal proteins (Giefing et al., 2008; Gierahn and Malley, 2011; Lu et al., 2018; Nabors et al., 2000; Schmid et al., 2011; Voß et al., 2018). So far, efforts in this direction have been focused on using surface-exposed pneumococcal proteins as these might be directly recognized by the host immune system. However, surface-exposed proteins typically show significant strain-to-strain sequence variability because of antigenic variation (Slager et al., 2018) rendering them prone to vaccine escape. In addition, since many surface proteins interact with human receptors, they might share structural homology to human proteins providing a concern related to autoimmune reactions in man.

Thus, there is an urgent need for an efficient pneumococcal vaccine which can cover most virulent pneumococcal strains and provide protection against superinfection.

### SUMMARY OF THE INVENTION

The present invention provides an immunogenic composition comprising at least one isolated antigenic polypeptide, with the amino acid sequence selected from the group comprising SEQ ID NO: 1, a fragment thereof, a variant thereof, and a combination of one or more thereof, wherein the isolated antigenic polypeptide is an intracellular polypeptide of S. pneumoniae.

Further provided is a nucleic acid sequence encoding at least one amino acid sequence of SEQ ID NO: 1, a fragment thereof, a variant thereof, and/or a combination of one or more thereof.

Further provided is a vaccine composition comprising i) an immunogenic composition of the invention, or ii) a nucleic acid of the invention, and an excipient, carrier, and/or adjuvant.

Further provided is an immunogenic composition of the invention, or a nucleic acid of the invention, or a vaccine composition of the invention, for use in the treatment and/or prevention of a disease caused by a Streptococcus infection, preferably by a Streptococcus pneumoniae infection.

Further provided is a method of treatment and/or prevention of a disease caused by a Streptococcus infection, preferably by a Streptococcus pneumoniae infection, the method comprising administering a therapeutically effective amount of an immunogenic composition, acid nucleic or vaccine of the present invention to a subject in need thereof.

Further provided is a kit comprising a therapeutically effective amount of i) an immunogenic composition of the invention, ii) a nucleic acid of the invention, or iii) a vaccine composition of the invention, for the treatment and/or prevention of a disease caused by a Streptococcus infection.

Also provided is a device comprising a therapeutically effective amount of i) an immunogenic composition of the invention, ii) a nucleic acid of anyone of the invention, or iii) a vaccine composition of the invention, for nasal administration.

### DESCRIPTION OF THE FIGURES

FIG. 1. Wild type pneumococci outcompete the lafB mutant in an influenza A virus pulmonary pneumococcal superinfection model. Wild type pneumococci and the mutants (ΔlafB and capsule-deficient Δcps) were first grown separately in vitro and were mixed in a 1:1 ratio prior to infection. Mice were first infected with 50 plaque-forming units (PFU) of murine-adapted H3N2 influenza A virus (IAV), followed 7 days later by intranasal infection of 1×10⁴ CFU of the mix of wild type and mutant pneumococci, as described previously (Liu et al., 2021). After 24 h following pneumococcal infection, bacteria were collected from the lung (left) or spleen (right) and plated to count the number of CFU. The competition index was calculated by dividing the number of recovered mutant bacteria by the number of recovered wild type bacteria. VL3200 is a strain with no gene deletion and acted as a negative control so a competition index of 1 was expected. The capsule of S. pneumoniae is a known virulence factor in this model (Liu et al., 2021), so the deletion mutant (Acps) worked as a control for a true virulence factor showing a competition index lower than 1. Horizontal bar indicates average. * (p<0.05), **, (p<0.01), *** (p<0.001), and **** (p<0.0001) indicate significantly different bacterial loads; “NS” means not significant, Kruskal-Wallis one-way ANOVA. This data shows that the lafB mutant is attenuated compared to the wild type.

FIG. 2. Reduced bacterial load of a lafB mutant in an influenza A virus pulmonary pneumococcal superinfection model. Mice were challenged with wild-type (D39V), lafB deletion mutant (ΔlafB) or a lafB deletion mutant complemented with lafB expressed from its native promoter at the ectopic ZIP locus (ΔlafB complemented) in the IAV superinfection model (Liu et al., 2021). Data showed the bacterial load (CFU) of lung (left) or spleen (right) at 24 h post pneumococcal infection. Each dot represents a single mouse. **** (p<0.0001) and *** (p<0.001) indicate significantly different bacterial loads, “NS” means not significant, Kruskal-Wallis one-way ANOVA. Horizontal bar indicates average. This experiment shows that LafB is a bona fide essential virulence factor.

FIG. 3. Purification of LafB recombinant protein from E. coli BL21. Shown is the purification of LafB with a Heparin column. S. pneumoniae lafB was cloned, expressed and purified in E. coli BL21 as described in the Methods. Shown are fractions collected of the elution from the heparin column. This shows that Streptococcus pneumoniae LafB can be purified untagged in high quantities to a very high purity from Escherichia coli.

FIG. 4. Antibody response induced by immunization with LafB protein. For intranasal immunization, 20 μg of LafB was used per dose with 2 μg of recombinant flagellin (FliC) as adjuvant. For subcutaneous immunization, 20 μg of LafB per dose with alum as adjuvant was used. The schedule for immunization of the intranasal route and subcutaneous route are the same. Primer vaccination performed on day 1, followed by booster vaccination on day 14. The serum was collected on day 28 from the immunized mice. The IgG and IgA antibody response induced by LafB was tested by ELISA with serum and bronchoalveolar lavage (BAL) collected from the immunized mice. Pneumovax is a CSP-based vaccine, here as negative control, and mice were immunized with 4 μg/mouse Pneumovax by subcutaneous route. “LafB by intranasal route” represents immunization with only LafB protein by intranasal route. “FliC-adjuvanted LafB by intranasal route” represents immunization with FliC-adjuvanted LafB by intranasal route. “Alum-adjuvanted LafB by subcutaneous route” represents immunization with alum-adjuvanted LafB by subcutaneous route. This experiment shows that LafB-vaccinated mice via the subcutaneous route generate a strong and specific IgG response, while this is not observed via the intranasal route.

FIG. 5. Mouse and rabbit serum of LafB-vaccinated animals recognize pneumococcal LafB as shown by Western blotting. Whole cell lysates of wild type S. pneumoniae D39V, lafB deletion mutant (ΔlafB), and the lafB complementation strain (ΔlafB, ZIP::Pnative-lafB) were used in the assay. (A) Samples blotting with mouse anti-serum. The anti-serum collected on day 28 from immunized mice, as described in FIG. 4, was diluted 1:500, and HRP conjugate goat-anti-mouse antibody was diluted 1:2500 as the secondary antibody. (B) Samples blotting with rabbit anti-serum. To raise the rabbit anti-lafB polyclonal antibody, a rabbit was immunized individually with 100 μg of purified LafB protein with a non-Freund adjuvant on day 0, 7, 10 and 18. The serum was collected on day 28. The collected anti-serum was diluted 1:500 and used as first antibody in the assay. The HRP conjugate goat-anti-rabbit antibody was diluted 1:5000 and used as the secondary antibody.

FIG. 6. Strong and specific T cells responses in LafB vaccinated mice. For intranasal immunization, 20 μg of LafB was used per dose, with 2 μg of recombinant flagellin FliC as adjuvant (Flagellin+LafB). For subcutaneous immunization, 20 μg of LafB per dose with alum as adjuvant (Alum+LafB), or with AS03 as adjuvant (AS03+LafB). Mice were immunized with 4 μg/mouse Pneumovax by subcutaneous route (Pneumo Vax). The schedule for immunization of the intranasal route and subcutaneous route are the same. Primer vaccination performed on day 1, followed by booster vaccination on day 14. The lung, spleen and lymph nodes were sampled for T cells on day 28 from the immunized mice. T-cells were stimulated ex vivo with either medium (negative control), LafB antigen or aCD3 (positive control). ELISA was used to assess Th1 (IFNg) response.

FIG. 7. The experiment was done the same as FIG. 6, except that ELISA was used to assess Th2 (IL-13) response.

FIG. 8. The experiment was done the same as FIG. 6, except that ELISA was used to assess Th17 (IL-17A) response.

FIG. 9. The experiment was done the same as FIG. 6, except that ELISA was used to assess Th17 or ILC3 (IL-22) response. This experiment, resulting in data shown in FIGS. 6-9, shows that the LafB antigen, in all tested formulations and administrations, generate T cell immunity in mice.

FIG. 10. Immunization with LafB increased the survival and promoted the gain of weight in the murine superinfection model. Mice were first infected with influenza A virus followed by challenge with 5×10⁴ CFU of serotype 2 strain Streptococcus pneumoniae D39V as described (Liu et al., 2021). Immunization with recombinant flagellin FliC-adjuvanted LafB (20 μg) by intranasal route; Pneumovax (4 μg) by subcutaneous route; alum-adjuvanted LafB (20 μg) by subcutaneous route; LafB (20 μg) by intranasal route without adjuvant; with vehicle as negative control (Mock) were tested. ** indicates significant difference (p<0.01) analyzed by Gehan-Breslow-Wilcoxon test.

FIG. 11. Western blotting with mouse anti-LafB serum towards different S. pneumoniae strains. The LafB protein from D39V (serotype 2), purified from E. coli (see FIG. 3) was the one used for immunization (subcutaneous with alum, see FIG. 4). Serum of vaccinated mice was used in Western blotting. Strains with different serotypes were included, including strains currently not included in PCV13 and PCV20 (e.g. serotypes 15A and 24F). 2 ng of purified LafB protein (LafB, 2 ng) was used here as a positive control for the reaction and a lafB deletion mutant (ΔlafB) was loaded as negative control. This experiment shows that LafB is highly conserved and serum of mice vaccinated with S. pneumoniae strain D39V LafB recognizes LafB in all tested pneumococcal serotypes as shown by Western blotting.

FIG. 12. Immunization with LafB from S. pneumoniae serotype 2 strain D39V increased the survival and promoted the gain of weight in the murine superinfection model challenged with serotype 15A and 24F pneumococci for which Pneumovax provides poor protection. Mice were first infected with influenza A virus followed by challenge with 5×10⁴ CFU of serotype 15A strain or with 1×10³ CFU of serotype 24F strain. Immunization with recombinant flagellin FliC-adjuvanted LafB (20 μg) by intranasal route; Pneumovax (4 μg) by subcutaneous route; or with vehicle as negative control (Mock) were tested. ** indicates significant difference (p<0.01) analyzed by Gehan-Breslow-Wilcoxon test.

FIG. 13. Phylogenetic tree of the homologs/variants of LafB (listed in Table 1). The phylogenetic tree is generated by Clustal Omega analysis (Madeira et al., 2019)

FIG. 14. The top hits of HHpred-search to identify similar structures as LafB.

FIG. 15. The model of LafB protein structure predicted by RoseTTAFold (Reference: https://doi.org/10.1126/science.abj8754). 5 models of LafB were predicted, all with confidence as 0.82. The 5 models were aligned by Crystallographic Object-Oriented Toolkit, Coot version 0.9.5. The different gray scales show the 5 different models

FIG. 16. LafB is predicted as a cytoplasmic protein with a positively charged domain as the part interacting with cell membrane. The electrostatic property of LafB was analyzed by UCSF ChimeraX version 1.2. The gray scales of the bottom panel indicate the charges on the surface of the LafB protein.

FIG. 17. LafB is an intracellular, membrane-associated protein in Streptococcus pneumoniae. (A) LafB was tagged with GFP at its N-terminus and expressed from its native locus. Phase contrast and GFP fluorescence images are shown. Scale bar is 2 μm. (B) LafB was tagged at its N- or C-terminus with the HiBit split luciferase and is under the control of an IPTG-inducible promoter from an ectopic locus. Only upon lysing of IPTG-induced cells in presence of the Nano-Glo Extracellular Detection System reagent, bioluminescence is produced, demonstrating that both N and C-terminus are intracellular.

FIG. 18. Preparation of nanoparticles of recombinant LafB-Halo protein. LafB-Halo protein without further intervention (1) was compared to a preparation in which the HaloTag Succinimidyl Ester was pre incubated for 30 minutes in buffer (2) and directly added to the LafB-Halo protein (3). This demonstrates the possibility of creating protein nanoparticles based on chemical connecting the polypeptides.

FIG. 19. Conjugation of LafB protein to S. pneumoniae serotype 6B capsule preparation. LafB proteins were coupled to capsule preparations using reductive amination. Samples were compared prior to further concentration (1), concentrated using a 30 kDA cutoff (2) as well as a 50 kDA cutoff (3) after the coupling and purification via gel filtration. A-C depict the Coomassie stained SDS-PAGE gels used to visualize the full protein content of gels not used for immunoblotting (A), used in LafB blotting (B) and in serotype 6B capsule blotting (C). Immunoblotting against LafB (D) and serotype 6B (E) demonstrate the possibility of conversion of LafB to high molecular weight species by coupling to S. pneumoniae capsule with the conservation of epitopes for antibodies raised against both constituents separately.

FIG. 20. Protection is mediated by IL-17A and CD4 T cells. C57BL/6, RoRgammac(t)-GfpTG or Il17a^{-/-} mice (n=4-10) were or left unvaccinated (mock) or immunized at days 1 and 14 with recombinant flagellin FliC-adjuvanted LafB by intranasal route and infected with IAV at day 28. (a) LafB-specific IL-17A secretion. Spleen, mediastinal lymph node and lung cells were collected at day 35 and stimulated 72h with LafB. IL-17A levels in supernatant were determined by ELISA. Plots represent values for individual mice as well as median and are representative of 2 independent experiments. Statistical significance (** P<0.01) was assessed by Mann-Whitney test compared to the mock group. (b). Protection is mediated by IL-17A. IL-17A-deficient (Il17a) mice (n=4-10) were or left unvaccinated (mock) or immunized at days 1 and 14 with recombinant flagellin FliC-adjuvanted LafB by intranasal route and infected with IAV at day 28. IAV-infected mice were challenged at day 35 with 5×10⁴ S. pneumoniae D39V strain and protection was assessed by monitoring survival. Protection is abolished in Il17a^{-/} mice. Statistical significance (*P<0.05, **p<0.01) was assessed by Mantel-Cox test compared to the mock group. (c) IL-17A producing cells in lung. IL-17A production by NKT cells (CD45+CD19−NK1.1−Gr1−CD11b−CD11c−αβTCR+TT+RoRγT+), type 3 innate lymphoid cells (ILC3) (CD45+CD19−NK1.1−Gr1−CD11b−CD11c−αβTCR-γδTCR-CD90+CD127+RoRγT+), γδ T cells (CD45+CD19−NK1.1−Gr1−CD11b−CD11c−αβTCR-γδTCR+RoRγT+), αβ T cells (CD45+CD19−NK1.1−Gr1−CD11b−CD11c−αβTCR+γδTCR−RoRγT+). Plots represent values for individual mice as well as median and are representative of 2 independent experiments. Statistical significance (*P<0.01) was assessed by Mann-Whitney test compared to the mock group. (d) Expression of CD69 marker on IL-17A producing αβ T cells. Representative of 2 independent experiments with 4 mice. (e) Protection is CD4 T cells dependent. To this end, at day 34 vaccinated and IAV infected mice were treated i.p. with CD4-specific depleting antibodies or control isotype and challenged 24 h later with 5×10⁴ S. pneumoniae D39v strain and protection was assessed by monitoring survival. Statistical significance (**p<0.01) was assessed by Mantel-Cox test compared to the mock group.

FIG. 21. Healthy human individuals have LafB-specific antibody and T cell responses. (a) DT- and LafB-specific IgG of plasma from healthy donors (n=127) were determined by ELISA. Plots represent values for individual people as well as median. (b) Western immunoblot assays of patient plasma to detect LafB-specific IgG. Plasma (n=4/group) with low and high absorbance at 450 nm in ELISA were analyzed by immunoblot. (c) PBMC from healthy donor were stimulated 5 days with LafB and IFNg production in supernatant were determined by ELISA. Plots represent values for individual wells and median from one representative donor from three independent donors. Statistical significance (*p<0.05, ***p<0.001) was assessed by one-way ANOVA Kruskal-Wallis test with Dunn's correction compared to the medium group.

### DESCRIPTION OF THE INVENTION

Although methods and materials similar or equivalent to those described herein can be used in the practice or testing of the present invention, suitable methods and materials are described below. All publications, patent applications, patents, and other references mentioned herein are incorporated by reference in their entirety. The publications and applications discussed herein are provided solely for their disclosure prior to the filing date of the present application. Nothing herein is to be construed as an admission that the present invention is not entitled to antedate such publication by virtue of prior invention. In addition, the materials, methods, and examples are illustrative only and are not intended to be limiting.

In the case of conflict, the present specification, including definitions, will control. Unless defined otherwise, all technical and scientific terms used herein have the same meaning as is commonly understood by one of skill in art to which the subject matter herein belongs. As used herein, the following definitions are supplied in order to facilitate the understanding of the present invention.

The term “comprise/comprising” is generally used in the sense of include/including, that is to say permitting the presence of one or more features or components. The terms “comprise(s)” and “comprising” also encompass the more restricted ones “consist(s)”, “consisting” as well as “consist/consisting essentially of”, respectively.

As used in the specification and claims, the singular form “a”, “an” and “the” include plural references unless the context clearly dictates otherwise.

As used herein, “at least one” means “one or more”, “two or more”, “three or more”, etc. . . . .

As used herein the terms “subject”/“subject in need thereof”, or “patient”/“patient in need thereof” are well-recognized in the art, and, are used interchangeably herein to refer to a mammal, including dog, cat, rat, mouse, monkey, cow, horse, goat, sheep, pig, camel, and, most preferably, a human. In some cases, the subject is a subject in need of treatment or a subject with a disease or disorder. However, in other aspects, the subject can be a normal subject. The term does not denote a particular age or sex. Thus, adult and newborn subjects, whether male or female, are intended to be covered. Preferably, the subject is a human, most preferably a human that might be at risk of suffering from a disease caused by a Streptococcus pneumoniae, or closely related subspecies, infection or a human suffering from a disease caused by a Streptococcus pneumoniae, or closely related subspecies, infection. In one aspect, the subject has been exposed to Streptococcus pneumoniae, wherein the subject is infected with Streptococcus pneumoniae, or wherein the subject is at risk of infection by Streptococcus pneumoniae.

According to the present invention, the disease caused by a Streptococcus pneumoniae infection is selected from the non-limiting group comprising pneumonia, sepsis, meningitis, otitis media, sacroiliitis (mitis), strangles, Streptococcal pharyngitis, abscesses, cellulitis, erysipelas, neonatal meningitis, endocarditis, urinary tract infections, dental caries, rhinitis, arthritis, mastoiditis, pelvic inflammatory disease, conjunctivitis, pericarditis, pleural empyema, prosthetic joint infection, vascular infection, uveitis, parapneumonic effusion and a combination of one or more thereof.

The term “vector”, as used herein, refers to a viral vector or to a nucleic acid (DNA or RNA) molecule such as a plasmid or other vehicle, which contains one or more heterologous nucleic acid sequence(s) of the invention and, preferably, is designed for transfer between different host cells. The terms “expression vector”, “gene delivery vector” and “gene therapy vector” refer to any vector that is effective to incorporate and express one or more nucleic acid(s) of the invention, in a cell, preferably under the regulation of a promoter. A cloning or expression vector may comprise additional elements, for example, regulatory and/or post-transcriptional regulatory elements in addition to a promoter.

The term “about,” particularly in reference to a given quantity, number or percentage, is meant to encompass deviations of plus or minus ten percent (+10). For example, about 5% encompasses any value between 4.5% to 5.5%, such as 4.5, 4.6, 4.7, 4.8, 4.9, 5, 4.1, 5.2, 5.3, 5.4, or 5.5.

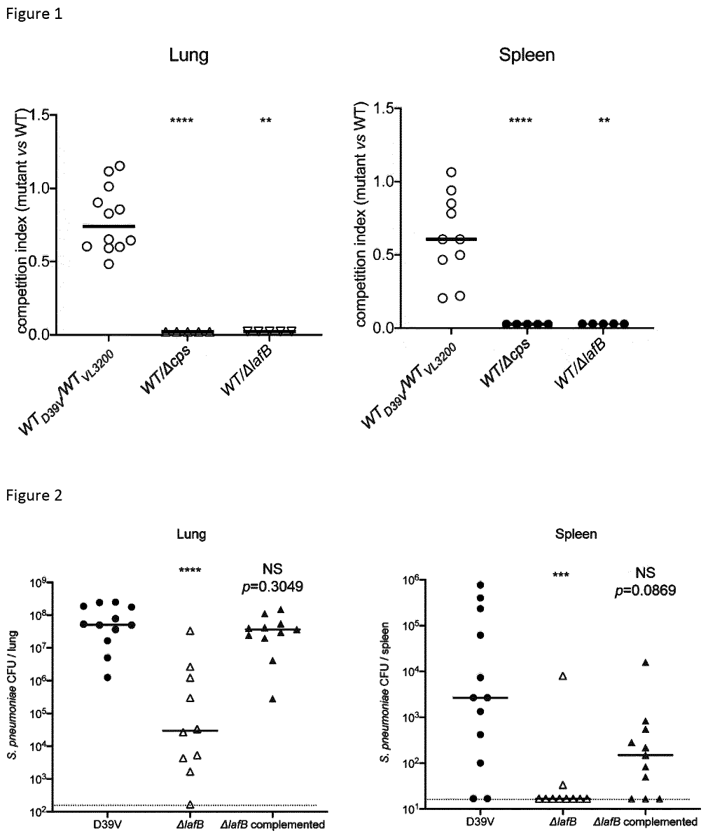

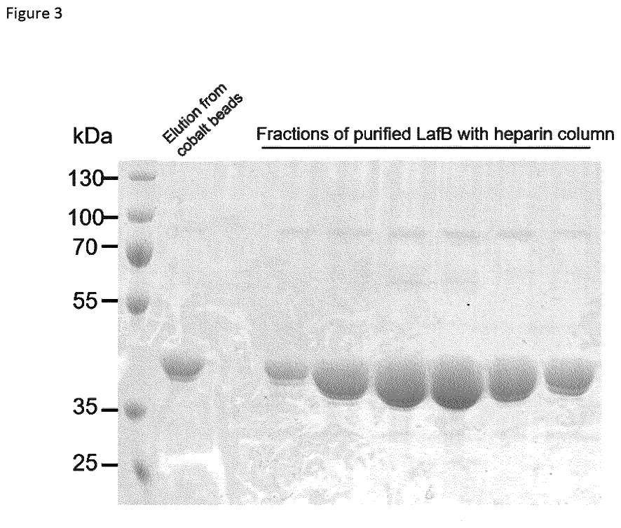

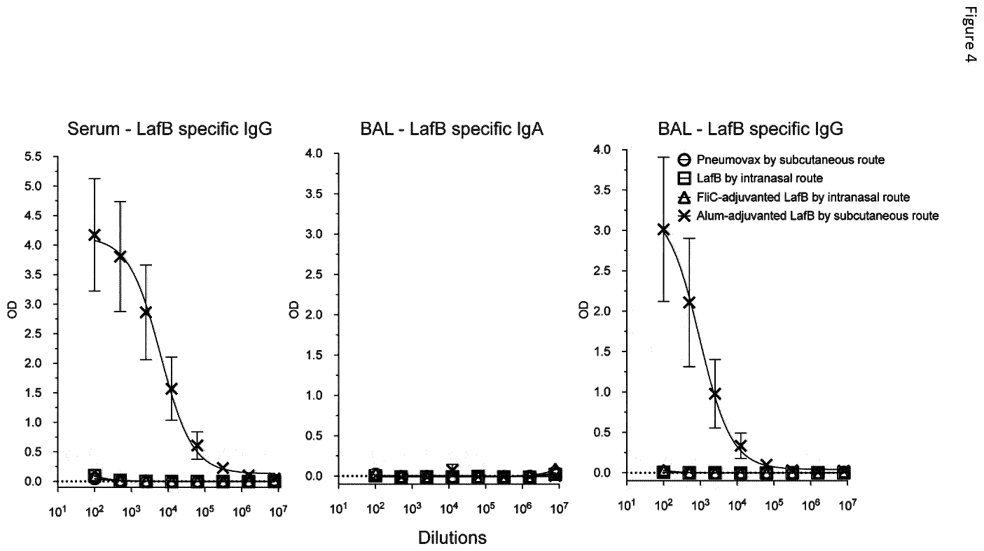

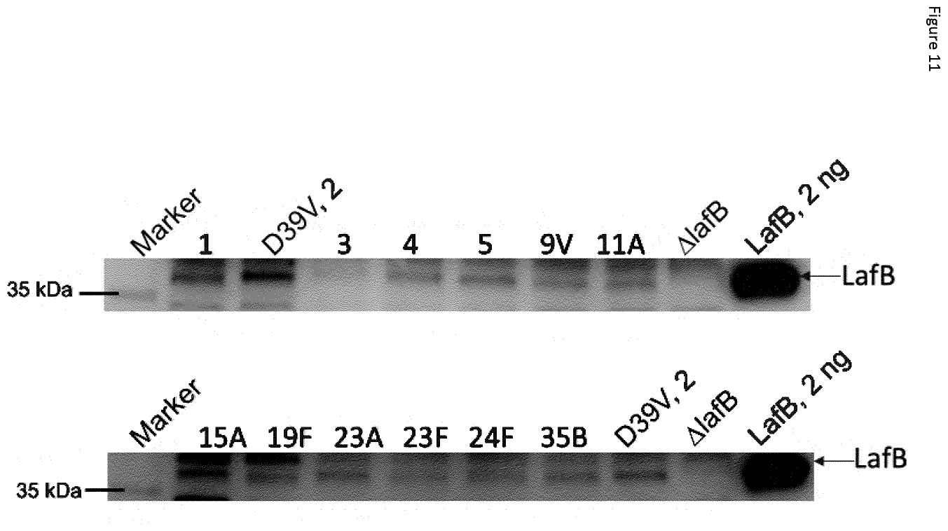

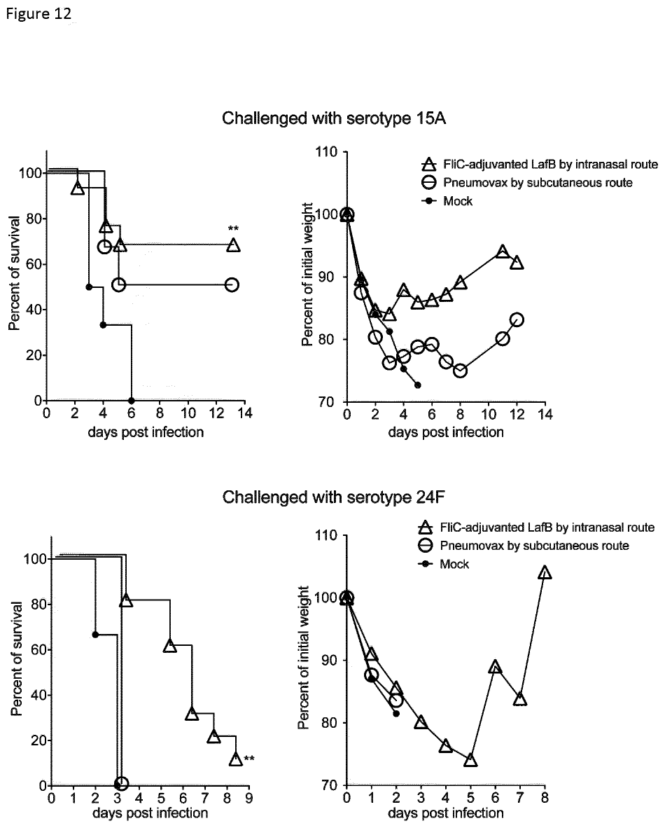

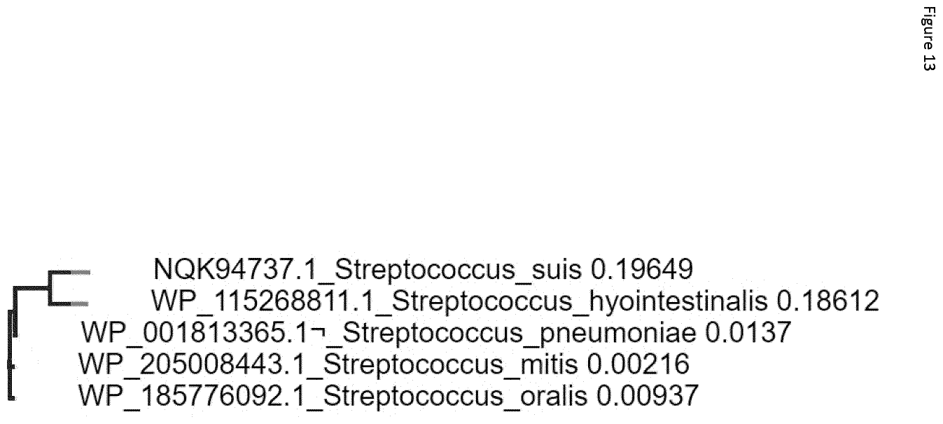

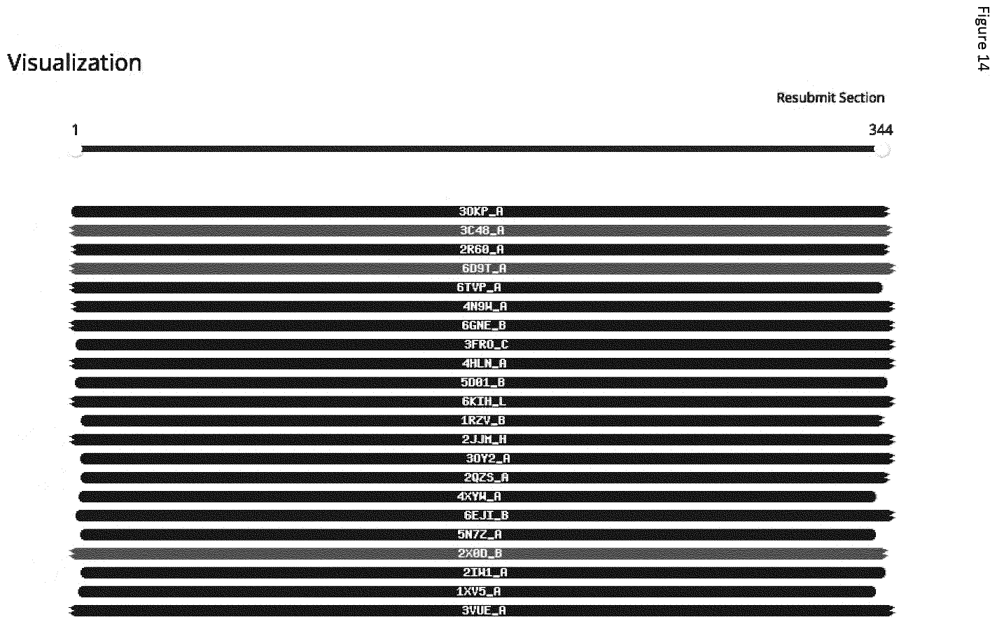

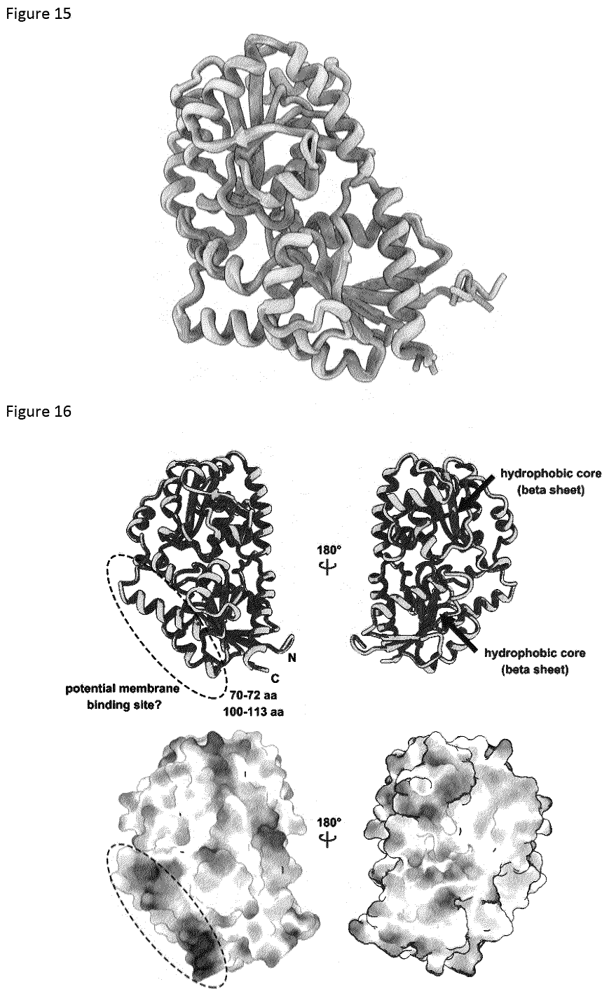

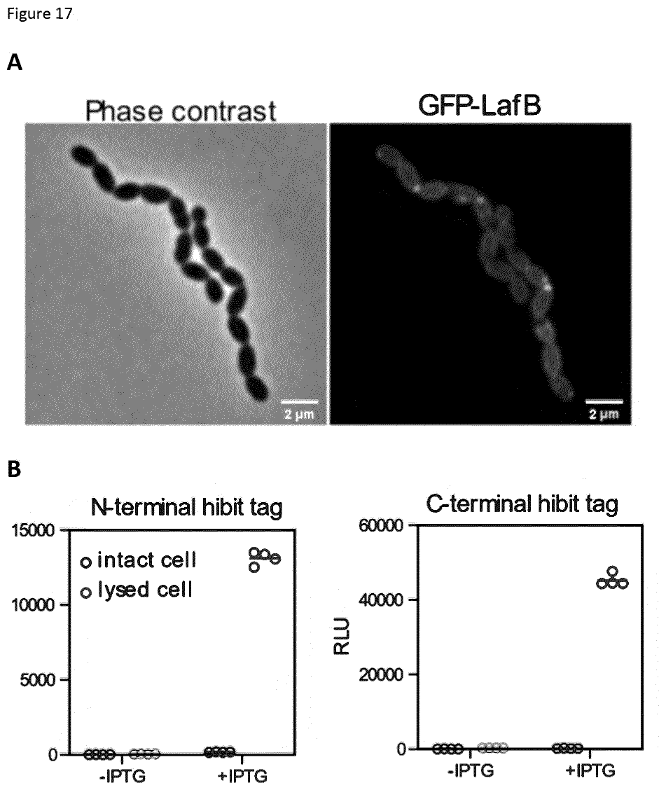

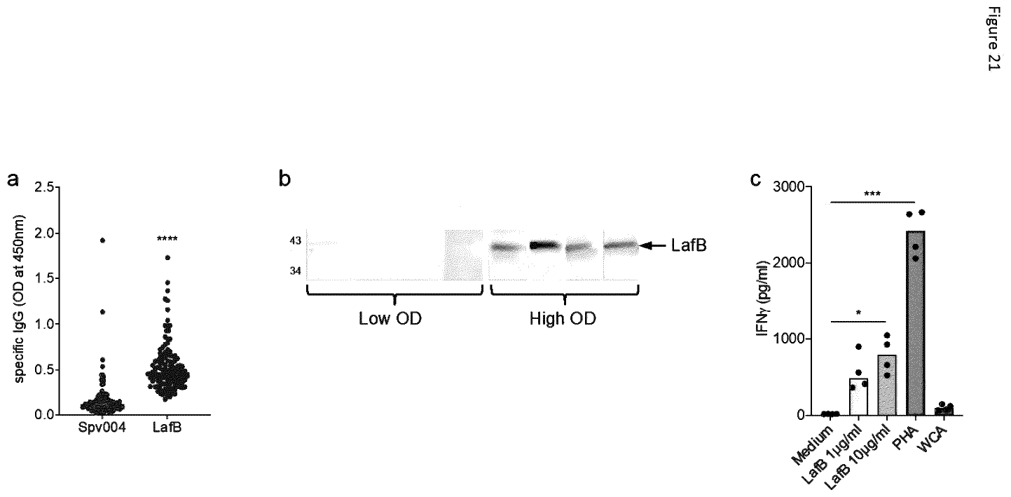

While focusing on the identification of potential universal pneumococcal vaccine antigens using CRISPRi-seq (Liu et al., 2021 CHM), the Inventors identified one promising vaccine candidate, LafB, as universal vaccine protecting from IPD. Whether intracellular pneumococcal proteins can act as potent protective antigens is currently unknown. Here, the Inventors identified a new virulence factor, LafB (FIGS. 1 and 2), that is highly conserved in pneumococci and closely related species (FIGS. 13 and 14), is shown to be intracellular (FIG. 17) and, when recombinantly produced and purified (FIG. 3) can provide protective immunity in a murine influenza-superinfection model (FIG. 10) across different pneumococcal serotypes (FIG. 12). In particularly, it is shown that strongest protection is via intranasal vaccination with recombinant flagellin FliC-adjuvanted LafB. It is shown that intranasal immunization with LafB protects in an IL-17A-dependent manner (FIG. 20), and that intranasal immunization with LafB protection requires CD4 cells that express RORgammaT and markers of tissue residency (FIG. 20). Finally, it is shown that healthy human individuals have LafB-specific antibody and T cell responses (FIG. 21).

Disclosed herein are immunogenic compositions. In one aspect, the present invention relates to an immunogenic composition comprising at least one isolated antigenic polypeptide, with the amino acid sequence selected from the group comprising SEQ ID NO: 1, a fragment thereof, a variant thereof, and a combination of one or more thereof.

Preferably, the isolated antigenic polypeptide is an intracellular polypeptide, more preferably an intracellular polypeptide of S. pneumoniae.

As used herein “Streptococcus pneumoniae”, “S. pneumoniae” or “pneumococcus” refers to a Gram-positive, alpha-hemolytic (under aerobic conditions) or beta-hemolytic (under anaerobic conditions), facultative anaerobic member of the genus Streptococcus. They are usually found in pairs (diplococci) and do not form spores and are nonmotile. S. pneumoniae can reside asymptomatically in healthy carriers typically colonizing the respiratory tract, sinuses, and nasal cavity. However, in susceptible individuals with weaker immune systems, such as the elderly and young children, the bacterium may become pathogenic and spread to other locations to cause disease.

S. pneumoniae can be differentiated from the viridans streptococci, some of which are also alpha-hemolytic, using an optochin test, as S. pneumoniae is optochin-sensitive. S. pneumoniae can also be distinguished based on its sensitivity to lysis by bile, the so-called “bile solubility test”. The encapsulated, Gram-positive, coccoid bacteria have a distinctive morphology on Gram stain, lancet-shaped diplococci. They have a polysaccharide capsule that acts as a virulence factor for the organism; more than 100 different serotypes are known (Ganaie et al. 2020, which is hereby incorporated by reference in its entirety), and these types differ in virulence, prevalence, and extent of drug resistance. Non-typeable, unencapsulated pneumococcal strains are also prevalent. In one aspect, the serotype is selected from the non-limiting group comprising serotype 1, 2, 6b, 15A and 24F, or a combination of or more thereof.

As used herein, the term “antigenic polypeptide” refers to a polypeptide that comprises at least one antigenic motif that induces, or is capable of inducing, an immune response. In the context of the present invention, the immune response is, preferably, towards a pneumococcal infection. In a particular aspect of the invention, the immune response is towards a Streptococcus pneumoniae infection.

As used herein, a “fragment” of one or more polypeptide sequence of the invention refers to a sequence containing less amino acids in length than the respective sequences of the invention while retaining the biological activity described herein. Preferably, a fragment of the polypeptide presenting an amino acid sequence as set forth in SEQ ID NO: 1, contains, at least 10%, 20%, 30%, 40%, 50%, 60%, 70%, 80%, or 90% of the entire length of the reference polypeptide sequence, preferably contiguous amino acid sequence of the reference polypeptide sequence.

In one aspect of the invention, a fragment of the polypeptide presenting an amino acid sequence as set forth in SEQ ID NO: 1, will have about 35 to 347, in particular at least 70, 105, 135, 170, 205, 240, 275, 305 or 345 amino acids, preferably contiguous amino acids. Most preferably, the fragment of SEQ ID NO: 1 comprises the amino acid sequence MEKKKLRIN and/or RKGIDDF. Most preferably, the fragment of SEQ ID NO: 1 comprises at least one antigenic motif that induces, or is capable of inducing, an immune response.

As used herein, the term “variant” refers to biologically active derivatives of a peptide or nucleic acid sequence. In general, the term peptide or polypeptide “variant” refers to molecules having a native sequence and structure with one or more additions, substitutions (generally conservative in nature) and/or deletions (e.g. splice variants, deletion of the one or more amino acids in first positions (such as Methionine) or last positions, . . . ), relative to the native molecule, so long as the modifications do not destroy biological activity and which are “substantially homologous” to the reference molecule. In general, the sequences of such variants are functionally, i.e. biologically, active variants and will have a high degree of sequence homology to the reference sequence, e.g., sequence homology of more than 50%, generally more than 60% or 70%, even more particularly 80% or more, such as at least 90% or 95% or more, when the two sequences are aligned. In one aspect, the reference sequence is SEQ ID NO. 1 or a fragment thereof.

Non-limiting examples of a variant of SEQ ID NO: 1 include one or more sequences selected from the group comprising SEQ ID NO: 2 (LafB-S. mitis), SEQ ID NO: 3 (LafB-S. oralis), SEQ ID NO: 4 (LafB-S. hyointestinalis), SEQ ID NO: 5 (LafB-S. suis) and a combination of one or more thereof.

TABLE 1

| SEQ ID NO: | Amino acid sequence |
|---|---|
| 1 | MEKKKLRINMLSSSEKVAGQGVSGAYRELVRLLHRAAKDQLIVTENLPIEADVTHFHTIDF |
| | PYYLSTFQK |
| | KRSGRKIGYVHFLPATLEGSLKIPFFLKGIVKRYVFSFYNRMEHLVVVNPMFIEDLVAAGI |
| | PREKVTYIP |
| | NFVNKEKWHPLPQEEVVRLRTDLGLSDNQFIVVGAGQVQKRKGIDDFIRLAEELPQITFIW |
| | AGGFSFGGM |
| | TDGYEHYKKIMENPPKNLIFPGIVSPERMRELYALADLFLLPSYNELFPMTILEAASCEAP |
| | IMLRDLDLY |
| | KVILEGNYRATAGREEMKEAILEYQANPAVLKDLKEKAKNISREYSEEHLLQIWLDFYEKQ |
| | AALGRK |
| 2 | MEKKKLRINMLSSSEKVAGQGVSGAYRELVRLLHRDAKDQLIVTENLPIEADVTHFHTIDF |
| | PYYLSTFQK |
| | KRSGRKIGYVHFLPDTLEGSLKIPFFLKGIVKRYVFSFYNRMEHLVVVNPMFIEDLVAAGI |
| | PREKVTYIP |
| | NFVNKEKWHPLPQEEVARLRTDLGLSDNQFIVVGAGQVQKRKGIDDFIRLAEELPQITFIW |
| | AGGFSFGGM |
| | TDGYERYKKIMENPPKNLIFPGIVSPERMRELYALADLFLLPSYNELFPMTILEAASCEAP |
| | IMLRDLDLY |
| | KVILEGNYRATADREEMKEAILEYQANPAALKDLKEKAKNISREYSEEHLLQIWLDFYEKQ |
| | AALGRK |
| 3 | MEKRGNFPLFSSSLSFLLLILFKENDIIVVMEKKKLRINMLSSSEKVAGQGVSGAYRELVR |
| | LLHRDAKDQ |
| | LIVTENLPIEADVTHFHTIDFPYYLSTFQKKRSGRKIGYVHFLPDTLEGSLKIPFFLKGIV |
| | KRYVFSFYN |
| | RMEHLVVVNPMFIEDLVAAGIPREKVTYIPNFVNKEKWHPLPQEEVARLRTELGLSDNQFI |
| | VVGAGQVQK |
| | RKGIDDFIRLAEELPEITFIWAGGFSFGGMTDGYERYKKIMENPPKNLIFPGIVSPEQMRE |
| | LYALADLFL |
| | LPSYNELFPMTILEAASCEAPIMLRDLDLYKVILEGNYRATADREEMKEAILEYQANPAAL |
| | KDLKEKAKN |
| | ISREYSEEHLLQIWLDFYEKQDALGRK |
| 4 | MTKEKIKVNMLSSSETVPGQGVSGAYRELMRLLKRDAREAIDLTENKRIKADITHYHTIDI |
| | PFYLSTFRK |
| | KRVGRRIGYVHFLPDTLQGSLRIPRIVQPIVAKYVTSFYNRMDHLVVVNPSFIEDLVAFGI |
| | DRDKITYIP |
| | NFVNSEKWFKPSAEDKSAVRKRYGWKDEDFVVLGAGQIQKRKGIDDFVTLAKTNPDIQFVW |
| | AGGFSFGGM |
| | TDGYDAYKKLMKNPPANLTFTGIVDADEMRQLYGACDLFLLPSFNELFPMTILEAAACGAP |
| | IMLRDLELY |
| | RIILEGMYLPASDVSKMSEAIRHLRTHPTELNHLQSQSEKISETYSEERLLETWLDFYRQQ |
| | ADLK |
| 5 | MSTKKLVINMLSSSEKVKGQGVSGAYRELMHLLRERGTTKLDIQEKLFVKADVTHYHTIDP |
| | IFFLTTFFK |
| | KRTGRRVGYVHFLPDTLEGSLQIPRFLTPLVSWYVITFYNRMDQLVVVNPSFIDDLVEYGI |
| | SRDKITYLP |
| | NFVNRDQWHPLSREEVQDVRKHYAISPNQFVVLGAGQVQRRKGIDDFAQLARECPDVTFVW |
| | AGGFSFGNM |
| | TDGHAEYKKLMEQPPKNLVFTGIVEAEEMLKLYNMADVFFLPSYSELFPMTILEAASCGTA |
| | IMLRDIDLY |
| | KVILEGMYLPVKNMDEMKTAIETLQSQPNQLVELTNKAGEISNYYSPEALLEKWLAFYQEQ |
| | ADLAKS |

The present invention further provides nucleic acid sequences. In one aspect, the nucleic acid sequence encodes at least one amino acid sequence selected from the group comprising SEQ ID NO: 1, a fragment thereof, a variant thereof, and a combination of one or more thereof.

The terms “nucleic acid,” “polynucleotide,” and “oligonucleotide” are used interchangeably and refer to a deoxyribonucleotide polymer (DNA, cDNA, . . . ) or ribonucleotide polymer (RNA, mRNA, . . . ) or to both, in linear or circular conformation, and in either single- or double-stranded form. For the purposes of the present disclosure, these terms are not to be construed as limiting with respect to the length of a polymer. The terms can encompass known analogues of natural nucleotides, as well as nucleotides that are modified in the base, sugar and/or phosphate moieties (e.g., phosphorothioate backbones). In general, an analogue of a particular nucleotide has the same base-pairing specificity, i.e., an analogue of A will base-pair with T. In a particular aspect, the ribonucleotide polymer is a mRNA, preferably a mRNA that comprises at least one chemical modification.

In one aspect, the RNA, e.g. the mRNA, comprises at least one chemical modification in at least one uracil. The at least one chemical modification is usually selected from pseudouridine, N1-methylpseudouridine, N1-ethylpseudouridine, 2-thiouridine, 4′-thiouridine, 5-methylcytosine, 5-methyluridine, 2-thio-1-methyl-1-deaza-pseudouridine, 2-thio-1-methyl-pseudouridine, 2-thio-5-aza-uridine, 2-thio-dihydropseudouridine, 2-thio-dihydrouridine, 2-thio-pseudouridine, 4-methoxy-2-thio-pseudouridine, 4-methoxy-pseudouridine, 4-thio-1-methyl-pseudouridine, 4-thio-pseudouridine, 5-aza-uridine, dihydropseudouridine, 5-methoxyuridine and 2′-O-methyl uridine.

In one aspect, the nucleic acid is an mRNA encoding at least one amino acid sequence selected from the SEQ ID NO: 1, a fragment thereof, a variant thereof, and a combination of one or more thereof. The mRNA can comprise at least one chemical modification as described above.

In another aspect, the nucleic acid is a DNA encoding at least one amino acid sequence selected from the SEQ ID NO: 1, a fragment thereof, a variant thereof, and a combination of one or more thereof.

In one aspect, the immunogenic composition disclosed in the present invention is to be used as a vaccine. In an aspect as above, the vaccine comprising the immunogenic composition disclosed in the present invention may be used for Streptococcus pneumoniae preventing and/or treating infection in a subject. Thus, in one aspect, the vaccine comprising the immunogenic composition disclosed in the present invention is to be used in the treatment and/or prevention of a disease caused by a Streptococcus infection, preferably by a Streptococcus pneumoniae infection. The disease caused by a Streptococcus infection, preferably by a Streptococcus pneumoniae infection is usually selected from the group comprising pneumonia, sepsis, meningitis, otitis media, sacroiliitis (mitis), strangles, Streptococcal pharyngitis, abscesses, cellulitis, erysipelas, neonatal meningitis, endocarditis, urinary tract infections, dental caries, rhinitis, arthritis, mastoiditis, pelvic inflammatory disease, conjunctivitis, pericarditis, pleural empyema, prosthetic joint infection, vascular infection, uveitis, parapneumonic effusion and a combination of one or more thereof.

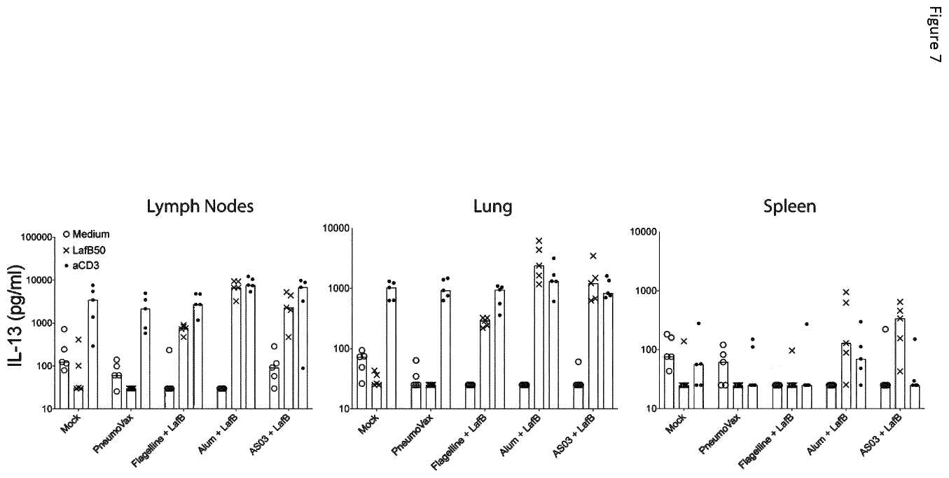

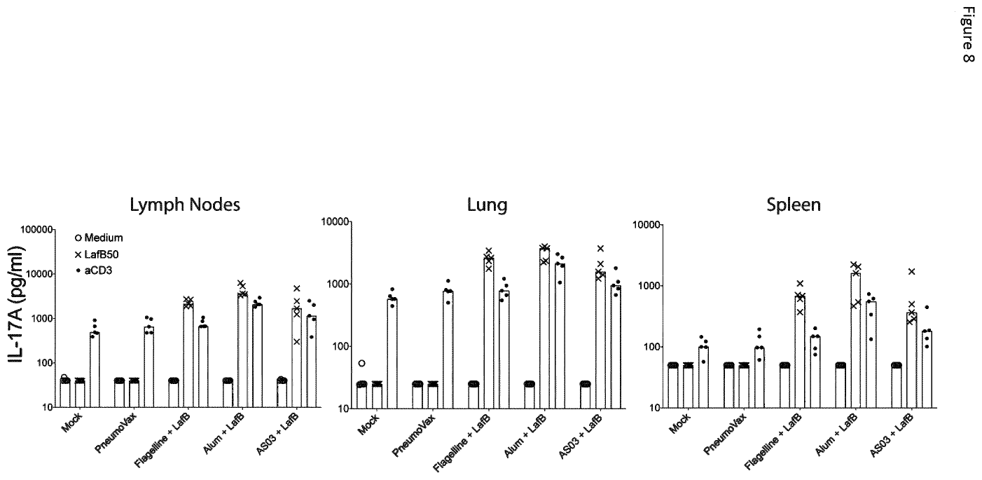

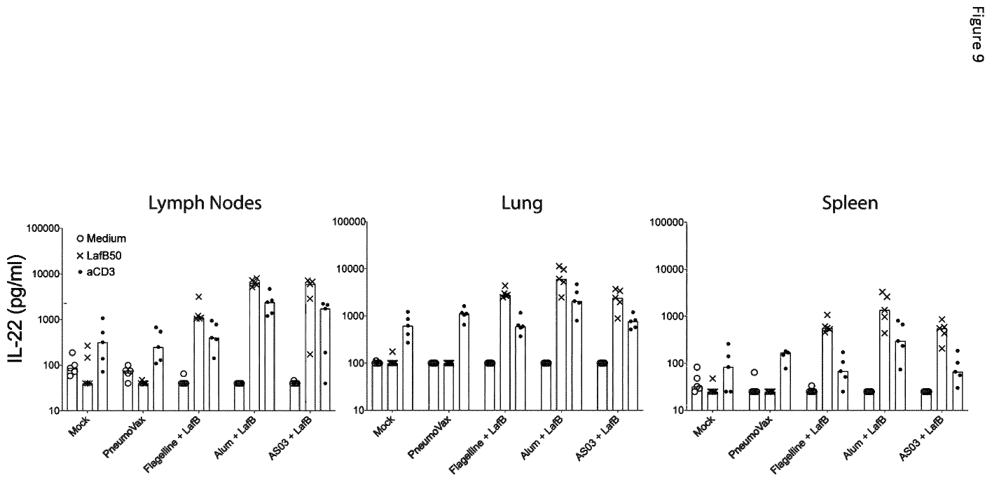

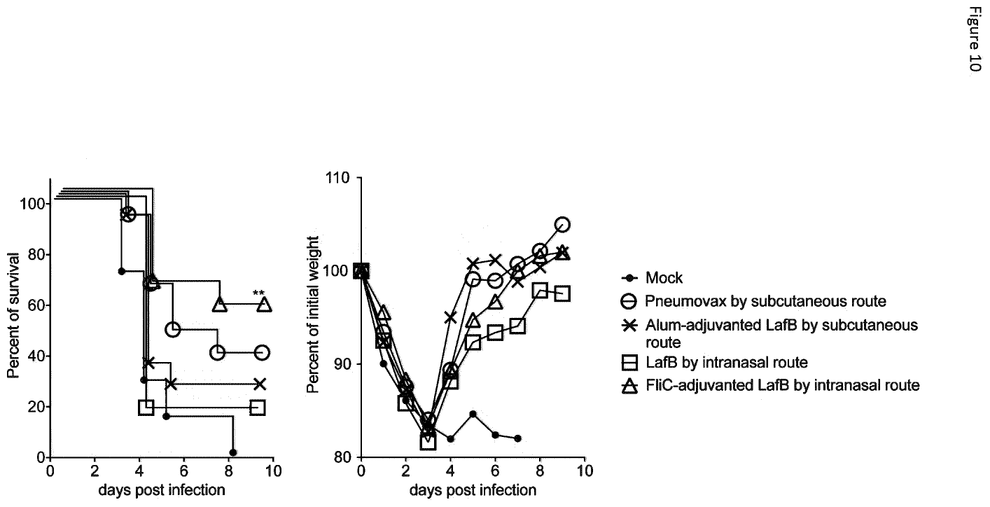

In one aspect, the vaccine triggers a T-cell specific response when administered to a subject (FIGS. 6-9).

In one aspect, the vaccine composition comprises i) an immunogenic composition of the invention, or ii) a nucleic acid of the invention, and an excipient, carrier, and/or adjuvant.

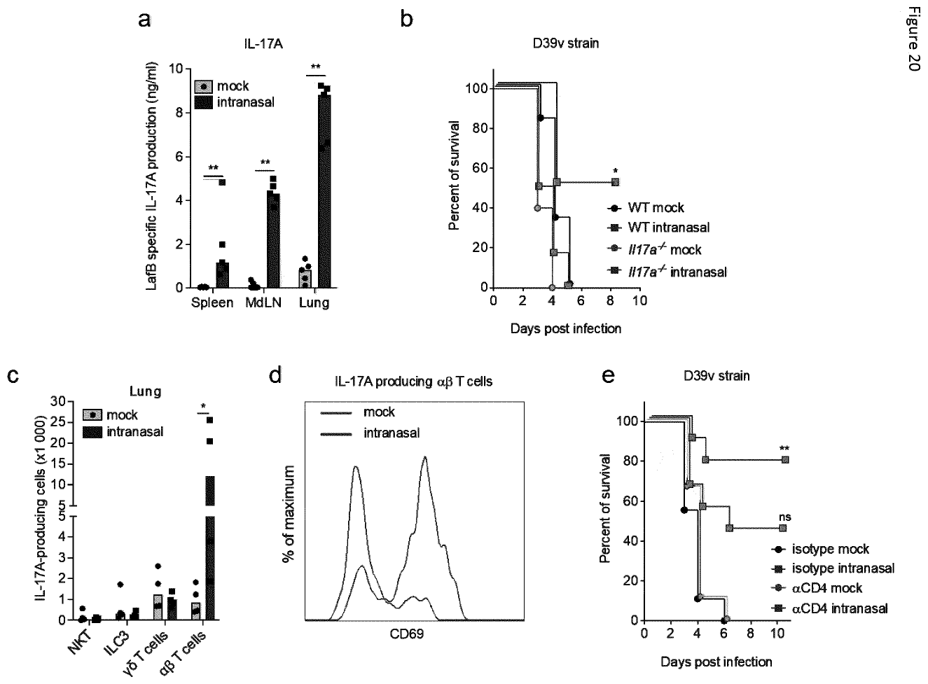

In one aspect, the immunogenic composition comprised in the vaccine and described herein is conjugated to a sugar, carrier and/or adjuvant (as shown in FIG. 19). Examples of conjugation processes are known from the art (see e.g. Möginger et al., 2016, which is hereby incorporated by reference in its entirety).

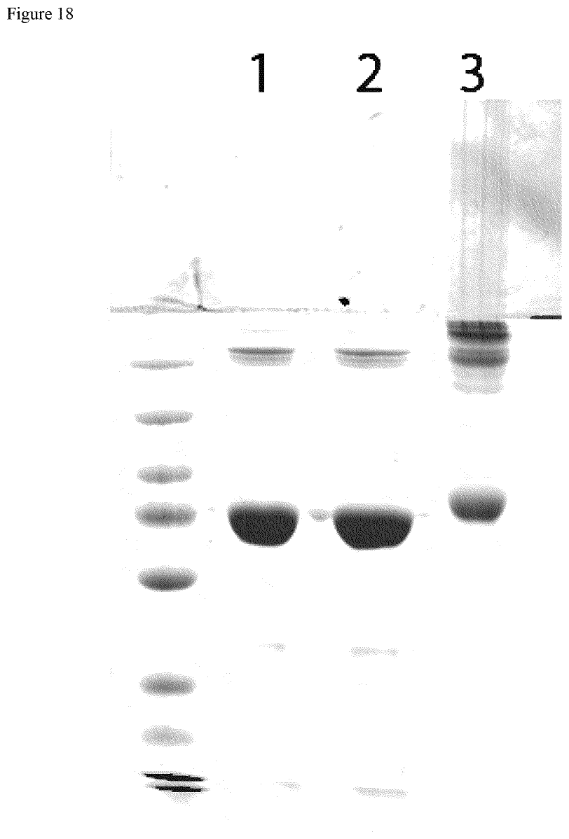

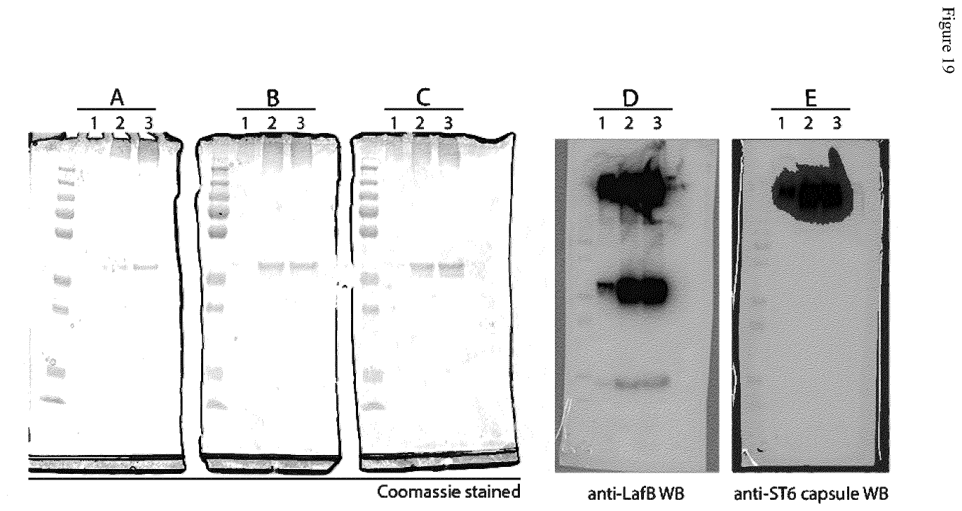

In one aspect, the immunogenic composition comprised in the vaccine and described herein is present as nanoparticles in which the protein vaccine is covalently attached to other protein vaccines via a chloroalkane linker using a modified haloalkane dehalogenase to form a large immunogenic network (as shown in FIG. 18). Examples of immunogenic nanoparticles are known from the art (see e.g. Curley and Putnam, 2022, 10.3389/fbioe.2022.867119, which is hereby incorporated by reference in its entirety).

Vaccine formulations of the invention may comprise one or more pharmaceutically acceptable carriers or excipients, which includes any excipient that does not itself induce a specific immune response. Suitable excipients include but are not limited to macromolecules such as proteins, saccharides, polylactic acids, polyglycolic acids, polymeric amino acids, amino acid copolymers, sucrose (Paoletti et al, 2001, Vaccine, 19:2118), trehalose, lactose and lipid aggregates (such as oil droplets or liposomes or lipid nanoparticles (LNP)). Such carriers are well known to the skilled artisan. Pharmaceutically acceptable excipients are discussed, e.g., in Gennaro, 2000, Remington: The Science and Practice of Pharmacy, 20th edition, ISBN: 0683306472).

Vaccine formulations of the invention may also comprise one or more pharmaceutically acceptable adjuvant. The term “adjuvant” refers to a compound or mixture which increases an immune response against an antigen. The adjuvant may enhance an immune response against an antigen exhibiting weak or insufficient immunogenicity and/or, may increase an antibody titer against an antigen and/or, may reduce an effective dose of an antigen for achieving an immune response in a subject, in case of single administration, as inducing no or weak antibody titer or cell mediated immune response. Thus, the adjuvant mostly plays a role of increasing an immune response, and this is known to those skilled in the art.

A suitable adjuvant enhancing the efficacy of a composition of the invention includes the followings, but it is not limited thereto:
a protein or a polysaccharide,
Cross-Reactive-Material-197 (CRM 197), tetanus toxoid, HaloTag, a polysaccharide (e.g. any serotype capsular polysaccharide, such as the serotype 1 capsular polysaccharide consisting of the following repeating trisaccharide: →4)-α-GalA-(1→3)-α-GalA-(1→3)-α-6dGalNAc4N-(1→), or a serotype 2 capsular polysaccharide consisting of the following repeating saccharide: →4)-β-D-Glcp-(1→3)-α-L-Rhap-(1→3)-α-L-Rhap-(1→3)-β-L-Rhap-(1→) with α-D-GlcpA-(1→6)-α-D-Glcp 1,2 linked to the second α-L-Rhap, or a serotype 6b capsular polysaccharide consisting of the following repeating saccharide→2)-α-D-Galp-(1→3)-α-D-Glcp-(1→3)-α-L-Rhap-(1→4)-D-Rib-ol-(5→P→), a flagellin (such as FliC), and a combination of one or more thereof,
An aluminum-based adjuvant, an agonist of toll-like receptors (TLRs), squalene-based adjuvant (such as AS03), monophosphoryl lipid A, synthetic nucleic acid sequences (such as CpG 1018), saponin-based adjuvant (such as QS-21), and a combination of one or more thereof (such as AS04).

Preferably, said aluminum-based adjuvant is selected from the group comprising aluminum phosphate, aluminum sulfate, aluminum hydroxide, and a combination of one or more thereof.

In one aspect, the immunogenic composition of the invention, the nucleic acid sequence encoding at least one amino acid sequence selected from the group comprising SEQ ID NO: 1, a fragment thereof, a variant thereof, and a combination of one or more thereof, or the vaccine comprising said immunogenic composition or acid nucleic of the present invention, is in a liquid form, preferably in an aqueous liquid form. The immunogenic composition, acid nucleic or vaccine of the present invention may comprise one or more kinds among buffer, salt, divalent cation, non-ionic detergent, cryoprotectant, e.g. sugar, and anti-oxidant, e.g. free radical scavenger and chelating agent, and any of various combinations thereof.

In one aspect, the immunogenic composition, acid nucleic or vaccine of the present invention comprises a buffer. In one aspect, the buffer has pKa of about 3.5 to about 7.5. In some aspects, the buffer is phosphate, succinate, histidine or citrate.

In one aspect, the immunogenic composition, acid nucleic or the vaccine of the present invention comprises a salt. In some aspects, the salt is selected from the group consisting of magnesium chloride, potassium chloride, sodium chloride and combinations thereof.

In one aspect, the immunogenic composition, acid nucleic or the vaccine of the present invention comprises a surfactant. The surfactant is selected from the group consisting of polyoxyethylene sorbitan fatty acid ester, polysorbate-80 (Tween 80), polysorbate-60 (Tween 60), polysorbate-40 (Tween 40) and polysorbate-20 (Tween 20), polyoxyethylene alkyl ether (including Brij 58, Brij 35, but not limited thereto), as well as other materials, for example, one or more kinds of non-ionic surfactants which include Triton X-100; Triton X-114, NP40, Span 85 and pluronic series of non-ionic surfactants (for example, pluronic 121), but not limited thereto. In a preferable aspect, the immunogenic composition comprises polysorbate-80 or polysorbate-20, preferably polysorbate-20. In a preferable aspect, the immunogenic composition comprises polysorbate-20 at a concentration of about 0.001% to about 2% (less than about 0.005% is preferable).

In one aspect, the immunogenic composition, acid nucleic or vaccine of the present invention are contained in a container prepared by glass, metal (for example, steel, stainless steel, aluminum, etc.) and/or polymers (for example, thermoplastic materials, elastomers, thermoplastic-elastomers). In one aspect, the container of the present invention is prepared by glass.

In one aspect, the present invention provides an injection filled with any one of the immunogenic compositions, acid nucleic or vaccine disclosed in the present invention. In one specific aspect, the injection is treated with silicon and/or is prepared by glass.

Preferably, the immunogenic composition, acid nucleic or vaccine disclosed in the present invention is administered by, intramuscular, subcutaneous, intravenous, intraperitoneal, intranasal, oral or intrathymic route of administration. More preferably, the immunogenic composition, acid nucleic or vaccine is administered by intramuscular or intranasal administration.

In one aspect, the vaccine composition comprises an immunogenic composition of the invention, and an excipient, carrier, and/or adjuvant, wherein the vaccine composition is for nasal administration and the adjuvant is a flagellin, preferably a recombinant flagellin FliC.

In one aspect, the vaccine composition comprises an immunogenic composition of the invention, and an excipient, carrier, and/or adjuvant, wherein the vaccine composition is for subcutaneous administration, and the adjuvant is an aluminum-based adjuvant, preferably alum.

In one aspect, the vaccine composition of the invention comprises an immunogenic composition of the invention, and an excipient, carrier, and/or adjuvant, wherein the vaccine composition is for intramuscular administration, and the adjuvant is an aluminum-based adjuvant, preferably alum.

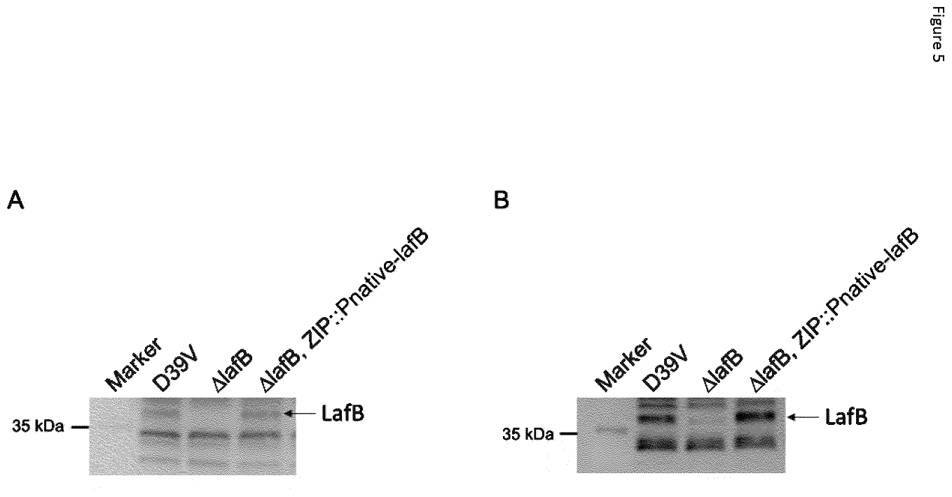

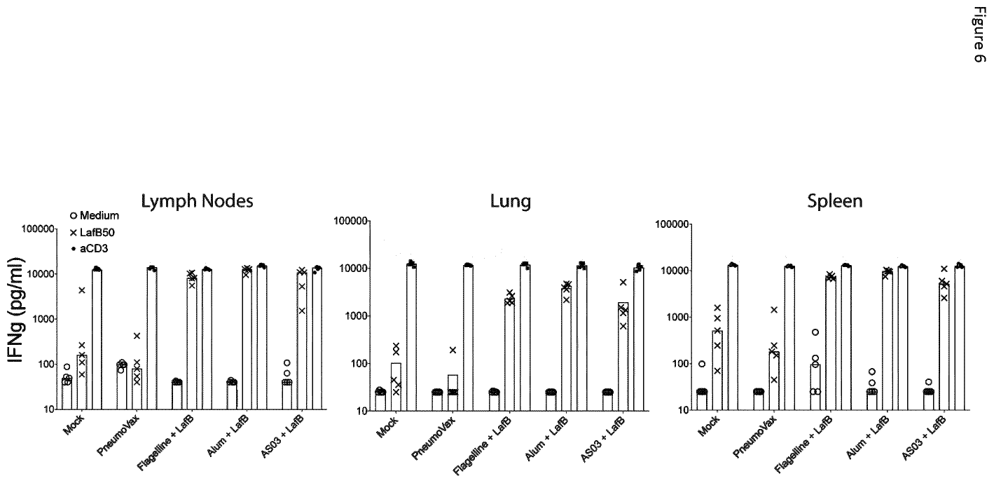

Preferably, the immunogenic composition, acid nucleic or vaccine disclosed in the present invention elicits specific T-cell responses, notably Th1, Th2 and Th17 as measured by IFNgamma, IL13 and IL-17A, respectively (see e.g. FIGS. 6-9). Preferably, the immunogenic composition, acid nucleic or vaccine disclosed in the present invention elicits antigen-specific IgG and IgM antibody production when administered via the intramuscular route (see e.g. FIG. 4).

Usually, the vaccine composition comprises a therapeutically effective amount of an immunogenic composition of the invention.

In some aspect, the vaccine composition contains about 0.01 μg and about 100 μg per dose or per administration of the at least one isolated antigenic polypeptide.

In some aspect, the vaccine composition contains about 0.1 ml to about 10 ml of the adjuvant composition.

In some aspect, the vaccine composition of the invention is administered to the subject more than once, preferably at least two times to the subject, with between 2-6 weeks in between each administration.

The present invention further provides the immunogenic composition of the invention, the nucleic acid of the invention, the vaccine composition of the invention, for use in the treatment and/or prevention of a disease caused by a Streptococcus infection, preferably by a Streptococcus pneumoniae infection.

The present invention further provides a method of treatment and/or prevention of a disease caused by a Streptococcus infection, preferably by a Streptococcus pneumoniae infection (FIGS. 10 and 12). The method comprises administering a therapeutically effective amount of an immunogenic composition, acid nucleic or vaccine disclosed in the present invention to a subject in need thereof.

As used throughout the description the terms “subject” and “subject in need thereof”, or “patient” and “patient in need thereof” are well-recognized in the art, and, are used interchangeably herein to refer to a mammal, including dog, cat, rat, mouse, monkey, cow, horse, goat, sheep, pig, camel, and, most preferably, a human. In some cases, the subject is a subject in need of treatment or a subject with a disease. However, in other aspects, the subject can be a normal subject. The term does not denote a particular age or sex. Thus, adult and newborn subjects, whether male or female, are intended to be covered. Preferably, the subject is a human, most preferably a human suffering from a disease caused by a Streptococcus infection or a human that might be at risk of suffering from a disease caused by a Streptococcus infection.

In one aspect, the administration route of a therapeutically effective amount of an immunogenic composition, acid nucleic or vaccine disclosed in the present invention is selected from the group comprising intramuscular, subcutaneous, intravenous, intraperitoneal, intranasal, oral or intrathymic.

In one aspect, the therapeutically effective amount of an immunogenic composition, acid nucleic or vaccine disclosed in the present invention, is administered to the subject in need thereof more than once, preferably at least two times to the subject in need thereof, with between 2-6 weeks in between each administration.

The present invention also encompasses a method for inducing an immune response against a disease caused by a Streptococcus infection, preferably by a Streptococcus pneumoniae infection. The method comprises administering a therapeutically effective amount of an immunogenic composition, acid nucleic or vaccine disclosed in the present invention to a subject in need thereof.

The present invention also provides a kit comprising a therapeutically effective amount of i) an immunogenic composition of the invention, ii) a nucleic acid of the invention, or iii) a vaccine composition of the invention, for the treatment and/or prevention of a disease caused by a Streptococcus infection.

A device comprising a therapeutically effective amount of i) an immunogenic composition of the invention, ii) a nucleic acid of the invention, or iii) a vaccine composition of the invention, for nasal administration.

The present invention further provides the use of an immunogenic composition, acid nucleic or vaccine disclosed in the present invention in the preparation of a medicament for the treatment and/or prevention of a disease caused by a Streptococcus infection, preferably by a Streptococcus pneumoniae infection.

The disclosure is further illustrated by the following examples. The examples below are non-limiting and are merely representative of various aspects of the disclosure.

### Examples

### Materials and Methods

### Identification of Novel Virulence Factors by CRISPRi-Seq

We developed a doxycycline-inducible CRISPRi system in Streptococcus pneumoniae, which enables both in vitro and in vivo studies (Liu et al., 2021). CRISPRi-seq screening was performed in both laboratory growth medium and in the murine superinfection model. The CRISPRi library was cultured in laboratory growth medium with or without doxycycline, the inducer of the system, for more than 20 generations. And then the bacteria are collected for gDNA isolation, followed by a one-step PCR for preparation of Illumina sequencing library. The screening with the superinfection model was started with flu infection on day 1. The mouse was infected intranasally with 50 plaque-forming units of H3N2 influenza A virus. On day 3 post-flu infection, the mice were fed with a control diet or a diet supplemented with doxycycline (200 mg/kg). On day 7, the mice were infected with 5×10⁵ CFU of the pneumococcal CRISPRi library. At 24 h post-pneumococcal infection, lungs of the mice were samples for extraction of genomic DNA of S. pneumoniae. Same as the screen in laboratory growth medium, the genomic DNA was then used for the one-step PCR for preparation of Illumina sequencing library (see (Liu et al., 2021) for more details). Virulence factors of the superinfection model were identified by comparing the fitness of specific genes in the laboratory growth medium to the superinfection model. Genes encoding putative virulence factors, such as LafB, were then mutated and mutant strains were either competed with wild type bacteria (FIG. 1) are tested individually (FIG. 2) for their impact on virulence.

Purification of LafB Protein from E. coli.

We cloned lafB with a CPD tag (Shen, 2014) into vector pLIBT7_A. See the appendix for the sequence of the vectors with the lafB, tagged with CPD (pLIBT7_A_lafB_CPDHisOld) or HALO (pLIBT7_A_lafB_HALO). The recombinant vectors were transformed into E. coli BL21. For expression of the protein, we first grew the E. coli BL21 with the vector in 500 ml of buffered TB medium to OD600 nm ˜0.6 at 37° C., 200 rpm. Buffered TB medium was made by first making buffer by autoclaving in 1 L of MQ water 2.4 g of KH2PO4 and 12.5 g of K2HPO4. TB medium contained 24 g of tryptone; 48 g of yeast extract; 10 ml of glycerol; in 900 ml of MQ water followed by sterilization via autoclaving. Finally, 900 ml of TB medium was mixed with 100 ml of the 10× Phosphate-buffered saline, to make 1 L of buffered TB medium. Then the cultures were cooled down to 16° C., and 0.5 mM IPTG (Isopropyl β-d-1-thiogalactopyranoside) was added into the culture to induce the expression of the protein overnight (˜14 hours). The bacteria were collected by centrifugation at 4° C., 5000 g. The pellets were resuspended with 75 ml of buffer (50 mM Tris-HCl, pH=7.5, 300 mM NaCl, 5% Glycerol, 25 mM Imidazole, 5 mM 2-mercaptoethanol, 1 mM PMSF, 750 Units of nuclease). E. coli cells were lysed by sonication. Cell lysates were centrifuged at 18,000 rpm, at 4° C. for 30 min. The supernatant was then collected for protein purification with cobalt beads. The protocol for purification of the CPD tagged protein is similar to the protocol published previously (Shen, 2014). Specifically, the supernatant was directly loaded onto cobalt beads, followed by washing with buffer (20 mM Tris, 100 mM NaCl) to remove the nonspecific bindings. We then used 25 ml of elution buffer (20 mM Tris, 100 mM NaCl with 2 mM inositol hexakisphosphate (InsP6)) to elute the protein. Addition of InsP6 activates the protease activity of CPD and the tag is cleaved off, so the final purified protein is tag free. The elution of LafB protein was further purified with Heparin column, and a gradient washing was made by mixing with buffer A1 (20 mM Tris, 100 mM NaCl) and buffer B1 (20 mM Tris, 1 M NaCl). The purified LafB was checked by SDS-PAGE (FIG. 3).

Coupling of LafB Protein to S. pneumoniae Capsule Preparation.

LafB proteins were dissolved in PBS to a final concentration of 1.2 mg/mL. Capsule preparations were oxidized via 10 mM Sodium (meta) periodate, dialyzed against PBS and then added to a final concentration of 0.1 mg/mL to the LafB protein. Reductive amination was enhanced by the addition of Sodium cyanoborohydride to a final concentration of 0.15 mg/mL. Coupling was undertaken at 4° C. over-night before gel filtration via a Superdex 200 10/300 gel filtration column.

### Creation of Nanoparticles of LafB-Halo Proteins.

LafB proteins tagged with the HaloTag were dissolved in PBS to a final concentration of 0.8 mg/mL. Bifunctional HaloTag Succinimidyl Ester (04) Ligand was added to a final concentration of 1.25 mM and incubated at room temperature for 30 minutes. Reactions were quenched by the addition of Tris-HCl pH 7.4 to a final concentration of 10 mM.

### Phase Contrast and Fluorescence Microscopy

S. pneumoniae cells were grown in C+Y medium pH=6.9 at 37° C. to an OD595 nm=0.1 without any inducer and diluted 100 times in fresh C+Y medium supplemented when appropriate with 100 μM IPTG. At OD 0.1 cells were harvested for fluorescence microscopy.

### Split-Luciferase HiBiT-Tag Detection System Assay

S. pneumoniae cells were grown in C+Y medium at 37° C. until OD595=0.2 and washed once with fresh C+Y medium. 5 μl of the Nano-Glo Extracellular Detection System reagent was added as specified in the manufacturer's instructions, and bioluminescence was detected in 96-wells plates with a Tecan Infinite 200 PRO luminometer at 37° C. Additionally, media and PBS samples were used as controls. Bioluminescence was measured right after the reagent addition. Two replicates for each time point and condition were tested.

Immunization of Mice with LafB (FIGS. 4,6,7,9)

For intranasal immunization, 20 μg of LafB was used per dose, with 2 μg of recombinant flagellin FliC as adjuvant. The recombinant flagellin primarily used in the present study derives from Salmonella enterica serovar Typhimurium FliC (accession number AAL20871) but is deleted from aminoacids 174 to 400 (Nempont et al. 2008). For subcutaneous immunization, 20 μg of LafB per dose with alum as adjuvant. The schedule for immunization of the intranasal route and subcutaneous route are the same. Primer vaccination performed on day 1, followed by booster vaccination on day 14. The serum was collected on day 28 from the immunized mice.

Mouse (C57BL/6j male or female) were immunized with 20 μg of LafB and 2 μg of recombinant flagellin FliC as adjuvant for intranasal route; or with 20 μg of LafB and alum as adjuvant for subcutaneous route. 4 μg of PneumoVax, or 0.4 μg of Prevenar were used per dose for subcutaneous route. Ovalbumin (OVA; as negative control) was used with 20 μg of OVA and 2 μg of recombinant flagellin FliC as adjuvant for intranasal route; or with 20 μg of OVA and alum as adjuvant for subcutaneous route. Vaccinations were performed twice, at day 1 and day 14. Serum and bronchoalveolar lavage (BAL) sampling for IgG, IgA and IgM determination was performed on day 28. Lung, spleen and lymph nodes sampling for T cells responses was performed on day 28. Immunized mice were challenged with 50 PFU H3N2 influenza A virus, typically on day 28. At day 35, the mice are further challenged with 4×10⁵ CFU S. pneumoniae D39V, or 5×10⁴ CFU S. pneumoniae serotype 15A, or 1×10³ CFU S. pneumoniae serotype 24F. Monitoring of infection, including survival and weight loss, were performed for 10 days after infection.

### REFERENCES

Avci, F. Y., Li, X., Tsuji, M., and Kasper, D. L. (2011). A mechanism for glycoconjugate vaccine activation of the adaptive immune system and its implications for vaccine design. Nat. Med. 17, 1602-1609.
Baek, M., et al. (2021). Accurate prediction of protein structures and interactions using a three-track neural network. Science. August 20; 373 (6557): 871-876. doi: 10.1126/science.abj8754. Epub 2021 Jul. 15.
Curley, S. M., and Putnam, D. (2022) Biological Nanoparticles in Vaccine Development. Front Bioeng Biotechnol. March 23; 10:867119. doi: 10.3389/fbioe.2022.867119. eCollection 2022.
Ganaie, F., Saad, J. S., McGee, L., van Tonder, A. J., Bentley, S. D., Lo, S. W., Gladstone, R. A., Turner, P., Keenan, J. D., Breiman, R. F., et al. (2020). A New Pneumococcal Capsule Type, 10D, is the 100th Serotype and Has a Large cps Fragment from an Oral Streptococcus. MBio 11.
Gennaro, A. L., Gennaro, and Gennaro, A. R. (2000). Remington: The Science and Practice of Pharmacy.
Giefing, C., Meinke, A. L., Hanner, M., Henics, T., Bui, M. D., Gelbmann, D., Lundberg, U., Senn, B. M., Schunn, M., Habel, A., et al. (2008). Discovery of a novel class of highly conserved vaccine antigens using genomic scale antigenic fingerprinting of pneumococcus with human antibodies. J. Exp. Med. 205, 117-131.
Gierahn, T., and Malley, R. (2011). Vaccines and compositions against Streptococcus pneumoniae.
Gonzalez, B. E., and Jacobs, M. R. (2013). The potential of human nasal colonization with Streptococcus pneumoniae as a universal pneumococcal vaccine. Am. J. Respir. Crit. Care Med. 187, 794-795.
Jirru, E., Lee, S., Harris, R., Yang, J., Cho, S. J., and Stout-Delgado, H. (2020). Impact of Influenza on Pneumococcal Vaccine Effectiveness during Streptococcus pneumoniae Infection in Aged Murine Lung. Vaccines 8.
Liu, X., Kimmey, J. M., Matarazzo, L., de Bakker, V., Van Maele, L., Sirard, J.-C., Nizet, V., and Veening, J.-W. (2021). Exploration of Bacterial Bottlenecks and Streptococcus pneumoniae Pathogenesis by CRISPRi-Seq. Cell Host Microbe 29, 107-120.e6.
Love, M. I., Huber, W., and Anders, S. (2014). Moderated estimation of fold change and dispersion for RNA-seq data with DESeq2. Genome Biol. 15, 550.
Lu, Y.-J., Oliver, E., Zhang, F., Pope, C., Finn, A., and Malley, R. (2018). Screening for Th17-Dependent Pneumococcal Vaccine Antigens: Comparison of Murine and Human Cellular Immune Responses. Infect. Immun. 86.
Madeira, F., Park, Y. mi, Lee, J., Buso, N., Gur, T., Madhusoodanan, N., Basutkar, P., Tivey, A. R. N., Potter, S. C., Finn, R. D., et al. (2019). The EMBL-EBI search and sequence analysis tools APIs in 2019. Nucleic Acids Res. 47, W636-W641.
McCullers, J. A. (2014). The co-pathogenesis of influenza viruses with bacteria in the lung. Nat. Rev. Microbiol. 12, 252-262.
Metzger, D. W., Furuya, Y., Salmon, S. L., Roberts, S., and Sun, K. (2015). Limited Efficacy of Antibacterial Vaccination Against Secondary Serotype 3 Pneumococcal Pneumonia Following Influenza Infection. J. Infect. Dis. 212, 445-452.
Möginger, U., Resemann, A., Martin, C. E., Parameswarappa, S., Govindan, S., Wamhoff, E.-C., Broecker, F., Suckau, D., Pereira, C. L., Anish, C., et al. (2016). Cross Reactive Material 197 glycoconjugate vaccines contain privileged conjugation sites. Sci. Rep. 6, 20488.
Nabors, G. S., Braun, P. A., Herrmann, D. J., Heise, M. L., Pyle, D. J., Gravenstein, S., Schilling, M., Ferguson, L. M., Hollingshead, S. K., Briles, D. E., et al. (2000). Immunization of healthy adults with a single recombinant pneumococcal surface protein A (PspA) variant stimulates broadly cross-reactive antibodies to heterologous PspA molecules. Vaccine 18, 1743-1754.
Nempont C, Cayet D, Rumbo M, Bompard C, Villeret V, Sirard J C. J Immunol. (2008). Deletion of flagellin's hypervariable region abrogates antibody-mediated neutralization and systemic activation of TLR5-dependent immunity. August 1; 181 (3): 2036-43. doi: 10.4049/jimmunol. 181.3.2036.
O'Brien, K. L., Wolfson, L. J., Watt, J. P., Henkle, E., Deloria-Knoll, M., McCall, N., Lee, E., Mulholland, K., Levine, O. S., Cherian, T., et al. (2009). Burden of disease caused by Streptococcus pneumoniae in children younger than 5 years: global estimates. Lancet Lond. Engl. 374, 893-902.
O'Toole, R. F. (2021). The interface between COVID-19 and bacterial healthcare-associated infections. Clin. Microbiol. Infect. Off. Publ. Eur. Soc. Clin. Microbiol. Infect. Dis.
Paoletti, L. C. (2001). Potency of clinical group B streptococcal conjugate vaccines. Vaccine 19, 2118-2126.
Pobre, K., Tashani, M., Ridda, I., Rashid, H., Wong, M., and Booy, R. (2014). Carrier priming or suppression: Understanding carrier priming enhancement of anti-polysaccharide antibody response to conjugate vaccines. Vaccine 32, 1423-1430.
Scelfo, C., Menzella, F., Fontana, M., Ghidoni, G., Galeone, C., and Facciolongo, N. C. (2021). Pneumonia and Invasive Pneumococcal Diseases: The Role of Pneumococcal Conjugate Vaccine in the Era of Multi-Drug Resistance. Vaccines 9.
Schmid, P., Selak, S., Keller, M., Luhan, B., Magyarics, Z., Seidel, S., Schlick, P., Reinisch, C., Lingnau, K., Nagy, E., et al. (2011). Th17/Th1 biased immunity to the pneumococcal proteins PcsB, StkP and PsaA in adults of different age. Vaccine 29, 3982-3989.
Shen, A. (2014). Simplified protein purification using an autoprocessing, inducible enzyme tag. Methods Mol. Biol. Clifton NJ 1177, 59-70.
Slager, J., Aprianto, R., and Veening, J.-W. (2018). Deep genome annotation of the opportunistic human pathogen Streptococcus pneumoniae D39. Nucleic Acids Res. 46, 9971-9989.
Voß, F., Kohler, T. P., Meyer, T., Abdullah, M. R., van Opzeeland, F. J., Saleh, M., Michalik, S., van Selm, S., Schmidt, F., de Jonge, M. I., et al. (2018). Intranasal Vaccination With Lipoproteins Confers Protection Against Pneumococcal Colonisation. Front. Immunol. 9, 2405.
Zhang, F., Lu, Y.-J., and Malley, R. (2013). Multiple antigen-presenting system (MAPS) to induce comprehensive B- and T-cell immunity. Proc. Natl. Acad. Sci. U.S.A 110, 13564-13569.
US20210154287 STREPTOCOCCUS PNEUMONIAE CAPSULAR POLYSACCHARIDES AND IMMUNOGENIC CONJUGATE THEREOF.

1. An immunogenic composition comprising at least one isolated antigenic polypeptide, with the amino acid sequence selected from the group consisting of SEQ ID NO: 1, a fragment thereof, a variant thereof, and a combination of one or more thereof, wherein the isolated antigenic polypeptide is an intracellular polypeptide of S. pneumoniae.

2. The immunogenic composition of claim 1, wherein the variant has at least 60% sequence homology with SEQ ID NO: 1, or with a fragment thereof.

3. The immunogenic composition of claim 1, wherein the variant of SEQ ID NO: 1 is selected from the group consisting of listing of SEQ ID NO: 2, SEQ ID NO: 3, SEQ ID NO: 4, SEQ ID NO: 5, a fragment thereof, and a combination of one or more thereof.

4. The immunogenic composition of claim 1, wherein the fragment of SEQ ID NO: 1 comprises the amino acid sequence MEKKKLRIN and/or RKGIDDF.

5. A nucleic acid sequence encoding the amino acid sequence of SEQ ID NO: 1, a fragment thereof, a variant thereof, and/or a combination of one or more thereof.

6. The nucleic acid sequence of claim 5, wherein the nucleic acid sequence is selected from the group consisting of a deoxyribonucleotides, a ribonucleotide polymer, a combination of deoxyribonucleotide and ribonucleotide polymer, in linear or circular conformation, a variant thereof and in either single- or double-stranded form.

7. The nucleic acid sequence of claim 6, wherein the ribonucleotide polymer is a mRNA.

8. The nucleic acid sequence of claim 7, wherein the mRNA comprises at least one chemical modification.

9. A vaccine composition comprising i) the immunogenic composition of claim 1, or ii) a nucleic acid encoding the amino acid sequence of SEQ ID NO: 1, a fragment thereof, a variant thereof, and/or combination of one or more thereof; and an excipient, carrier, and/or adjuvant.

10. The vaccine composition of claim 9, wherein said immunogenic composition is conjugated to a sugar, a carrier and/or an adjuvant.

11. The vaccine composition of claim 9, wherein said vaccine composition is administered by; intramuscular, subcutaneous, intravenous, intraperitoneal, intranasal, oral or intrathymic route of administration.

12. The vaccine composition of claim 9, wherein said vaccine composition triggers a T-cell specific response when administered to a subject.

13. The vaccine composition of claim 9, wherein said carrier and/or adjuvant is a protein or a polysaccharide.

14. The vaccine composition of claim 9, wherein said carrier and/or adjuvant is selected from the group consisting of CRM 197, tetanus toxoid, HaloTag, APEX2, BioID2, TurboID, a polysaccharide, a flagellin, and a combination of one or more thereof.

15. The vaccine composition of claim 14, wherein said polysaccharide is pneumococcal capsule.

16. The vaccine composition of claim 38, wherein said serotype 1 capsular polysaccharide consists of the following repeating trisaccharide: →4)-α-GalA-(1→3)-α-GalA-(1→3)-α-6dGalNAc4N-(1→).

17. The vaccine composition of claim 38, wherein said serotype 2 capsular polysaccharide consists of the following repeating saccharide: →4)-β-D-Glcp-(1→3)-α-L-Rhap-(1→3)-α-L-Rhap-(1→3)-β-L-Rhap-(1→) with α-D-GlcpA-(1→6)-α-D-Glcp 1,2 linked to the second α-L-Rhap.

18. The vaccine composition of claim 38, wherein said serotype 6b capsular polysaccharide consists of the following repeating saccharide →2)-α-D-Galp-(1→3)-α-D-Glcp-(1→3)-α-L-Rhap-(1→4)-D-Rib-ol-(5→P→).

19. The vaccine composition of claim 9, wherein said adjuvant is selected from the group consisting of an aluminum-based adjuvant, an agonist of toll-like receptors (TLRs), squalene-based adjuvant, monophosphoryl lipid A, synthetic nucleic acid sequences, saponin-based adjuvant, and a combination of one or more thereof.

20. The vaccine composition of claim 19, wherein said aluminum-based adjuvant is selected from the group consisting of aluminum phosphate, aluminum sulfate, aluminum hydroxide, and a combination of one or more thereof.

21. A vaccine composition comprising the immunogenic composition of claim 1, and an excipient, carrier, and/or adjuvant, wherein the vaccine composition is formulated for nasal administration and the adjuvant is a flagellin.

22. A vaccine composition comprising the immunogenic composition of claim 1, and an excipient, carrier, and/or adjuvant, wherein the vaccine composition is formulated for subcutaneous administration, and the adjuvant is an aluminum-based adjuvant.

23. A vaccine composition comprising the immunogenic composition of claim 1, and an excipient, carrier, and/or adjuvant, wherein the vaccine composition is formulated for intramuscular administration, and the adjuvant is an aluminum-based adjuvant.

24. The vaccine composition of claim 9, wherein the at least one isolated antigenic polypeptide is present in an amount of about 0.01 μg to about 100 μg per dose or per administration.

25. The vaccine composition of claim 9, wherein the adjuvant composition is present in an amount of about 0.1 ml to about 10 ml.

26. The vaccine composition of claim 9, wherein the vaccine composition is administered to the subject more than once.

27. The vaccine composition of claim 26, wherein the vaccine composition is administered at least two times to the subject, with 2-6 weeks in between each administration.

28. (canceled)

29. (canceled)

30. A method of treatment of a disease caused by a Streptococcus infection, the method comprising administering to a subject in need thereof a therapeutically effective amount of:
i) the immunogenic composition of claim 1,
ii) a nucleic acid sequence encoding the amino acid sequence of SEQ ID NO. 1, a fragment thereof, a variant thereof, and/or a combination of one or more thereof, or
iii) a vaccine composition comprising i) the immunogenic composition of claim 1, or ii) the nucleic acid encoding the amino acid sequence of SEQ ID NO: 1, a fragment thereof, a variant thereof, and/or a combination of one or more thereof; and an excipient, carrier, and/or adjuvant.

31. The method of treatment according to claim 30, wherein the disease is caused by a Streptococcus pneumoniae infection.

32. The method of treatment according to claim 30, wherein the disease is selected from the group consisting of pneumonia, sepsis, meningitis, otitis media, sacroiliitis (mitis), strangles, Streptococcal pharyngitis, abscesses, cellulitis, erysipelas, neonatal meningitis, endocarditis, urinary tract infections, dental caries, rhinitis, arthritis, mastoiditis, pelvic inflammatory disease, conjunctivitis, pericarditis, pleural empyema, prosthetic joint infection, vascular infection, uveitis, parapneumonic effusion and a combination of one or more thereof.

33. The method of treatment according to claim 30, wherein the immunogenic composition, the nucleic acid or the vaccine composition is administered by an administration route comprising intramuscular, subcutaneous, intravenous, intraperitoneal, intranasal, oral or intrathymic administration.

34. The method of treatment according to claim 30, wherein the vaccine composition is administered to the subject in need thereof more than once.

35. The method of treatment according to claim 34, wherein the vaccine composition is administered at least two times to the subject in need thereof, with 2-6 weeks in between each administration.

36. A kit comprising a therapeutically effective amount of:
i) the immunogenic composition of claim 1,
ii) a nucleic acid sequence encoding the amino acid sequence of SEQ ID NO: 1, a fragment thereof, a variant thereof, and/or a combination of one or more thereof, or
iii) a vaccine composition comprising i) the immunogenic composition of claim 1, or ii) the nucleic acid encoding the amino acid sequence of SEQ ID NO: 1, a fragment thereof, a variant thereof, and/or a combination of one or more thereof; and an excipient, carrier, and/or adjuvant;
and instructions for the treatment of a disease caused by a Streptococcus infection.

37. A device comprising a therapeutically effective amount of:
i) the immunogenic composition of claim 1,
ii) a nucleic acid sequence encoding the amino acid sequence of SEQ ID NO: 1, fragment thereof, a variant thereof, and/or a combination of one or more thereof, or
iii) a vaccine composition comprising i) the immunogenic composition of claim 1, or ii) the nucleic acid encoding the amino acid sequence of SEQ ID NO: 1, a fragment thereof, a variant thereof, and/or a combination of one or more there; and an excipient, carrier, and/or adjuvant, wherein the device is adapted for nasal administration.

38. The vaccine composition of claim 15, wherein the pneumococcal capsule is a serotype 1 capsular polysaccharide, a serotype 2 capsular polysaccharide, a serotype 6b capsular polysaccharide, or a combination of one or more thereof.
